(19) United States
(12) Patent Application Publication
(10) Pub. No.: US 2023/0365516 A1
(43) Pub. Date: Nov. 16, 2023

(54) COMPOUNDS FOR CANCER THERAPY AND IMAGING

(71) Applicant: Washington University, St. Louis, MO (US)

(72) Inventors: SAMUEL ACHILEFU, St. Louis, MO (US); DOROTA GRABOWSKA, St. Louis, MO (US); KRISHNA SHARMAH GAUTAM, St. Louis, MO (US); PARTHA KARMAKAR, St. Louis, MO (US)

(21) Appl. No.: 18/016,235

(22) PCT Filed: Jul. 14, 2021
(86) PCT No.: PCT/US2021/041637

§ 371 Date: Jan. 13, 2023

Related U.S. Application Data
(60) Provisional application No. 63/051,681, filed on Jul. 14, 2020.

(51) Int. Cl. C07D 271/12; A61K 31/69; G01N 33/574; A61P 35/00

(52) U.S. Cl. CPC C07D 271/12; A61K 31/69; A61P 35/00; G01N 33/57484

(57) ABSTRACT

Among the various aspects of the present disclosure is the provision of compositions and methods for targeted treatment and detection of cancers. In particular, the present disclosure is directed to a compounds are from the benzofurazan, and nitrobenzofurazan family of compounds. The compounds inhibit tumor proliferation, induce regression of tumor growth or prevent tumor survival. In another embodiment, the compounds are labeled or conjugated with atoms or group of atoms that illuminate their distribution in cells and living organisms, the signals of which can be detected using imaging systems.

### CROSS REFERENCE TO RELATED APPLICATIONS

This application claims the benefit of U.S. Provisional Application No. 63/051,681, filed Jul. 14, 2020 the disclosure of which is herein incorporated by reference in its entirety.

### GOVERNMENTAL RIGHTS

This invention was made with government support under CA199092 awarded by the National Institutes of Health. The government has certain rights in the invention.

### FIELD OF THE TECHNOLOGY

The disclosed subject matter relates generally to cancer therapy and cancer detection. More particularly, the present disclosure relates to compositions and methods for treating or imaging a cancerous condition.

### BACKGROUND

Cancer detection and treatment are hindered by the inability to differentiate between cancer cells and normal cells. Improved tools for cancer or tumor imaging are needed for earlier detection and treatment of cancers. Molecular recognition of tumor cells would facilitate guided surgical resection. In order to improve surgical resection, targeted imaging tools must specifically label tumor cells, not only in the main tumor but also along the edge of the tumor and in the small tumor cell clusters that disperse throughout the body.

Cancer cells undergo selection pressure when exposed to chemotherapy or radiotherapy, leading to genetic mutations that allow them to evade treatment, metastasize, and relapse. Despite efforts to develop novel therapies that inhibit multiple signaling pathways, only a small number of cancer types exhibit sustainable remission. One such cancer type that exhibits frequent relapse is multiple myeloma (MM), a disease of the plasma cells that originates in the bone marrow. In the last 5 years alone, the US Food and Drug Administration has approved six new drugs for MM patients; yet these drugs have limited survival benefits, with some extending lives for only a couple of months. Despite these advances, a significant number of patients have primary refractory disease and fail to respond to induction treatments. Furthermore, after an initial response, the transformed cancer becomes resistant to current therapies. As a result, nearly all patients diagnosed with MM will eventually relapse and die from the disease.

Although curative measures are desirable, they have proved elusive. However, novel therapies that can prolong remission or prevent relapse, and which can use different mechanisms of action from current therapies, are needed to treat resistant phenotypes and increase survival while retaining the quality of life.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A shows TEM image of blank liposome. FIG. 1B shows the hydrodynamic diameter of HSA-LS1090 NPs measured by dynamic light scattering (DLS), number distribution curve. FIG. 1C shows the Structure of LLP2A. FIG. 1D shows the structure of LS288

FIG. 2A shows MM1.S cell viability with different formulations of LS1090 at 24h incubation. FIG. 2B shows the cytotoxicity of HSA-LS1090 against PMBC and MM1.S cell lines at different effective concentrations of LS1090.

FIGS. 3A-3D show in vivo therapeutic effect of HSA-LS 1090 against disseminated MM1.S-GFP Luc in Fox Chase SCID Beige mice. FIG. 3A shows the tumor progression over time monitored by BLI. The arrows on X axis indicates days of therapy. FIG. 3B shows the comparative tumor burden in groups of mice at the end of a week 6 post therapy. FIG. 3C shows the comparative tumor burden in groups of mice at the end of a week 7 post therapy. FIG. 3D shows the comparative tumor burden in groups of mice at the end of a week 9 post therapy. (Statistical analysis performed by two-tailed unpaired t-test).

FIGS. 4A-4G show the identification and characterization of LS1090 as a potential drug candidate for MM. FIG. 4A shows the schematic of the high-throughput screening assay used to identify LS1090 as a potential drug candidate against human MM cells. FIG. 4B shows the structure of LS1090. FIG. 4C shows a dose-response EC₅₀ plots for LS1090 against different human MM cell lines FIG. 4D shows a hydrodynamic diameter of HSA-LS1090 NPs measured by DLS, volume percentage distribution. FIG. 4E shows the hydrodynamic diameter of HSA-LS1090 NPs measured by DLS, intensity percentage distribution. FIG. 4F shows the cytotoxicity of LS1090 and HSA-LS1090 against PBMCs (effective LS1090 concentration in free LS1090 and in HAS-LS1090 is shown). FIG. 4G shows the cytotoxicity of HAS-LS1090 against MM1.S PBMCs (effective LS1090 concentration in HAS-LS1090 is shown) percentage of necrotic tissue in harvested tumors following H&E staining 10 days after irradiation.

FIGS. 5A-5H show LS1090 induced apoptosis in MM1.S cells. FIG. 5A shows apoptosis and necrosis processes monitored in real time at EC₅₀ concentration of LS1090 in MM1.S cells. FIG. 5B shows apoptosis and necrosis processes monitored in real time at EC₅₀ concentration of LS1090 in U266 cells FIG. 5C shows apoptosis and necrosis processes monitored in real time at EC₅₀ concentration of HSA-LS1090 in MM1.S cells. Results are expressed as fluorescence units (RFU; necrosis/plasma membrane integrity) or luminescence units (RLU; apoptosis/PtSer:AnxV binding); n = 4 for each condition. FIG. 5D shows LS1090 stimulation of caspase 3/7 depends on ERK activation in MM1.S cells. FIG. 5E shows caspase inhibitor (Z-VAD-fmk) protects MM1.S cells from LS1090 induced apoptosis; n = 4 for each condition. FIG. 5F shows LS1090 activates ERK in a time-dependent manner. FIG. 5G shows LS1090 shows minimal effect on p53 and Bcl-2 expressions. FIG. 5F shows LS1090 stimulates Bax expression.

FIGS. 6A-6B show LS1090 overcomes the protective effects of IL-6 and IGF-1. FIG. 6A shows MM1.S cells were incubated with LS1090 for 24 h in the presence of IL-6. FIG. 6B shows IGF-1 compared to the LS1090-untreated control; n=4 for each condition.

FIGS. 7A-7D show LS1090 enhances BTZ toxicity in MM.1S cells. FIG. 7A shows BTZ but not LS1090 inhibits chymotrypsin-like activity in MM.1S cells assessed with chymotrypsin-like activity assay at 0.1 µM and 1.0 pM, respectively. FIG. 7B shows the EC₅₀ of BTZ improved against MM1.S cells with increasing LS1090 concentration compared to the EC50 of BTZ alone. FIG. 7A shows isobologram analysis demonstrates the cytotoxic effects of treating MM1.S cells with a combination of LS1090 and BTZ. The CI is a quantitative measure of the degree of drug interaction, with a CI < 1 indicating synergy, a CI = 1 indicating additive effect, and a CI > 1 indicating antagonism. FIG. 7D shows the concentrations of BTZ and LS1090 combinations used in the isobologram analysis.

FIGS. 8A-8D show In vivo imaging and assessment of LS1090 therapeutic effects in MM mouse models. FIG. 8A shows the therapeutic effect of HSA-LS1090 and BTZ in disseminated MM1.S-GFP Luc bearing Fox-Chase SCID Beige mice. The arrows indicate the treatment regimen. The p values were calculated by Mann-Whitney test comparing the untreated and HSA-LS1090 treatment groups. FIG. 8B shows representative BLI image of the untreated and HSA-LS1090 treated groups on week 9 post inoculation (Color scale, p/s/cm2/sr = photon/second/cm2/steradian). FIG. 8C shows survival proportions for untreated, HSA-LS1090 and BTZ treated groups of mice. FIG. 8D shows change in body weight of mice from different groups during the course of the study.

FIGS. 9A-9B shows the effect of LS1090 on different cancer cell lines. FIG. 9A shows the LS1090 effect on human multiple myeloma cell lines in comparison to Bortezomib. FIG. 9B shows LS1090 effect on different cancer cell lines. Cells were treated with LS1090 for 24h, followed by assessment of viability using CellTitler-Glow assay. n=4 for each condition.

FIG. 10A shows the structure of LS1090 along with IC₅₀ values against different MM cell lines. FIG. 10B shows the absorption spectra of LS1090. FIG. 10C shows the emission spectra of LS1090. FIG. 10D shows the IC₅₀ curve for LS1090 against MM1.S cell line. FIG. 10E shows the IC₅₀ curve for LS1090 against OPM2 cell line. FIG. 10F shows the IC₅₀ curve for LS1090 against U266 cell line.

FIG. 11 shows IC₅₀ and calculated ClogP of representative analogues of LS1090 were similar to that of LS1090.

FIG. 12 shows a schematic of Example 3.

### DETAILED DESCRIPTION

Compositions comprising one or more benzofurazan-, or nitrobenzofurazan-derived molecules and methods of use are provided herein. The present disclosure is based, at least in part, on the discovery that the disclosed compounds inhibit tumor proliferation, induce regression of tumor growth and/or prevent tumor survival. In some embodiments, the compounds are labeled or conjugated with atoms or group of atoms that illuminate their distribution in cells and living organisms, the signals of which can be detected using imaging systems. Moreover, the disclosed compositions are found to provide unexpected synergistic effects when combined with standard-of-care therapeutics (e.g. bortezomib). Accordingly, the present disclosure also provides methods of treating a tumor or cancer.

Discussed below are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular component is disclosed and discussed and a number of modifications that can be made to a number of molecules of the component are discussed, specifically contemplated is each and every combination and permutation of the component and the modifications that are possible unless specifically indicated to the contrary. Thus, if components A, B, and C are disclosed as well as a component D, E, and F and an example of a combination composition, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Various aspects of the invention are described in further detail in the following sections.

### 1. Compostions

One aspect of the present disclosure encompasses benzofurazan-, or nitrobenzofurazan-derived molecules (e.g. compounds of formula (I)). In various embodiments the compounds can be synthesized by methods disclosed herein. The compounds disclosed herein may be modified to improve potency, bioavailability, solubility, stability, handling properties, or a combination thereof, as compared to an unmodified version. Thus, in another aspect, a composition of the disclosure comprises modified compound of formula (I). In still another aspect, a composition of the disclosure comprises a prodrug of compound disclosed herein.

A composition of the present disclosure may comprise one or more active agents. In some embodiments, an active agent may be an agent to treat, or reduce a cancer. In some embodiments, treating or reducing a cancer may comprise slowing the growth of a cancer cell. In some embodiments, treating or reducing a cancer may include killing a cancer cell. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents, or antioxidants.

Other aspects of the invention are described in further detail below.

### (A) Benzofurazan-, and Nitrobenzofurazan-derived Compounds

In general, the compounds detailed herein include compounds comprising a benzofurazan or a nitrobenzofurazan structure as diagrammed below.

Provided herein are compounds comprising Formula (I) and salts (e.g., pharmaceutically acceptable salts) thereof:

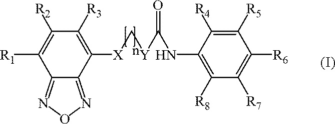

wherein X and Y are independently selected from O, S, NH Se, (OC₂H₄)₁₋₅;
wherein R¹, R², R³, R⁴, R⁵, R⁶, R⁷, and R⁸ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur; and
n is an integer from 1-5.

In an embodiment, a compound of Formula (I) comprises any of the preceding compounds of Formula (I), wherein R₁ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₁ is —NO₂.

In another embodiment, a compound of Formula (I) comprises any of the preceding compounds of Formula (I), wherein R₂ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₂ is H.

In still another embodiment, a compound of Formula (I) comprises any of the preceding compounds of Formula (I), wherein R₃ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, -OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₃ is H.

In yet another embodiment, a compound of Formula (I) comprises any of the preceding compounds of Formula (I), wherein R₄ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₄ is H.

In still yet another embodiment, a compound of Formula (I) comprises any of the preceding compounds of Formula (I), wherein R₅ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₅ is H.

In another embodiment, a compound of Formula (I) comprises any of the preceding compounds of Formula (I), wherein R₆ is selected hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₆ is H.

In still another embodiment, a compound of Formula (I) comprises any of the preceding compounds of Formula (I), wherein R₇ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₇ is CF₃. In another preferred embodiment, a compound of Formula (I) comprises any of the preceding compounds of Formula (I), wherein R₇ is CBr₃. In still another preferred embodiment, a compound of Formula (I) comprises any of the preceding compounds of Formula (I), wherein R₇ is CCl₃. In yet another preferred embodiment, a compound of Formula (I) comprises any of the preceding compounds of Formula (I), wherein R₇ is OMe. In another preferred embodiment, a compound of Formula (I) comprises any of the preceding compounds of Formula (I), wherein R₇ is OEt. In still another preferred embodiment, a compound of Formula (I) comprises any of the preceding compounds of Formula (I), wherein R₇ is OtBu. In still another preferred embodiment, a compound of Formula (I) comprises any of the preceding compounds of Formula (I), wherein R7 is a halogen (e.g., I, F, Br, Cl, including radioisotopes thereof).

In yet another embodiment, a compound of Formula (I) comprises any of the preceding compounds of Formula (I), wherein R₈ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (I) comprises any of the preceding compounds of Formula (I), wherein R₈ is H.

In still yet another embodiment, a compound of Formula (I) comprises any of the preceding compounds of Formula (I), wherein X is selected from the group O, S, NH Se, (OC₂H₄)₁₋₅. In a preferred embodiment, a compound of Formula (I) comprises any of the preceding compounds of Formula (I), wherein X is S. In another preferred embodiment, a compound of Formula (I) comprises any of the preceding compounds of Formula (I), wherein X is O. In still another preferred embodiment, a compound of Formula (I) comprises any of the preceding compounds of Formula (I), wherein X is NH.

In another embodiment, a compound of Formula (I) comprises any of the preceding compounds of Formula (I), wherein Y is selected from O, S, NH Se, (OC₂H₄)₁₋₅. In a preferred embodiment, a compound of Formula (I) comprises any of the preceding compounds of Formula (I), wherein Y is S. In another preferred embodiment, a compound of Formula (I) comprises any of the preceding compounds of Formula (I), wherein Y is O. In still another preferred embodiment, a compound of Formula (I) comprises any of the preceding compounds of Formula (I), wherein Y is NH.

In still another embodiment, a compound of Formula (I) comprises any of the preceding compounds of Formula (I), wherein n is an integer from 1-5. In a preferred embodiment, a compound of Formula (I) comprises any of the preceding compounds of Formula (I), wherein n is 2.

In one aspect, a compound of formula (I) comprises any of the preceding compounds of Formula (I), wherein R₇ is not CF₃.

Particularly useful compounds of formula (I) are those in Table 1

TABLE 1

| | |
|---|---|
| Compound Code | Structure |
| LS1090 | 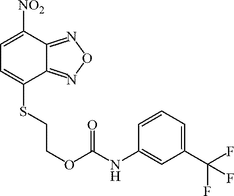 [SMILES: O=C(Nc1cccc(C(F)(F)F)c1)OCCSc1ccc([N+](=O)[O-])c2nonc12] |
| LS1110 | 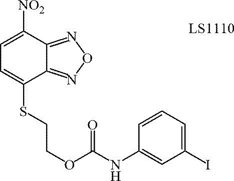 |
| LS1111 | 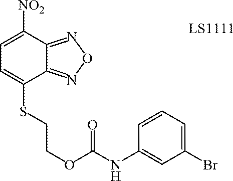 |
| LS1112 | 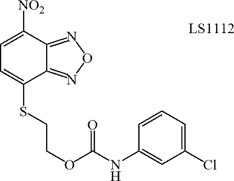 |
| LS1113 | 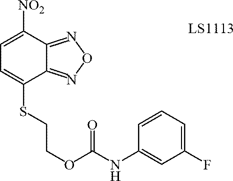 |

In another embodiment, provided herein are compounds comprising Formula (II) and salts (e.g., pharmaceutically acceptable salts) thereof:

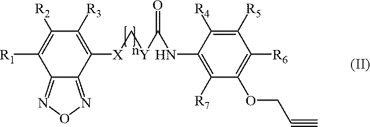

wherein X and Y are independently selected from O, S, NH Se, (OC₂H₄)₁₋₅,
wherein R₁, R₂, R₃, R₄, R₅, R₆, and R₇ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur; and
n is an integer from 1-5.

In an embodiment, a compound of Formula (II) comprises any of the preceding compounds of Formula (II), wherein R₁ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₁ is NO₂.

In another embodiment, a compound of Formula (II) comprises any of the preceding compounds of Formula (II), wherein R₂ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₂ is H.

In still another embodiment, a compound of Formula (II) comprises any of the preceding compounds of Formula (II), wherein R₃ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₃ is H.

In yet another embodiment, a compound of Formula (II) comprises any of the preceding compounds of Formula (II), wherein R₄ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₄ is H.

In still yet another embodiment, a compound of Formula (II) comprises any of the preceding compounds of Formula (II), wherein R₅ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₅ is H.

In another embodiment, a compound of Formula (II) comprises any of the preceding compounds of Formula (II), wherein R₆ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₆ is H.

In yet another embodiment, a compound of Formula (II) comprises any of the preceding compounds of Formula (II), wherein R₇ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (II), wherein R₇ is H.

In still yet another embodiment, a compound of Formula (II) comprises any of the preceding compounds of Formula (II), wherein X is selected from the group O, S, NH Se, (OC₂H₄)₁₋₅. In a preferred embodiment, a compound of Formula (II) comprises any of the preceding compounds of Formula (II), wherein X is S. In another preferred embodiment, a compound of Formula (II) comprises any of the preceding compounds of Formula (II), wherein X is O. In still another preferred embodiment, a compound of Formula (II) comprises any of the preceding compounds of Formula (II), wherein X is NH.

In another embodiment, a compound of Formula (II) comprises any of the preceding compounds of Formula (II), wherein Y is selected from the group O, S, NH, Se, (OC₂H₄)₁₋₅. In a preferred embodiment, a compound of Formula (II) comprises any of the preceding compounds of Formula (II), wherein Y is S. In another preferred embodiment, a compound of Formula (II) comprises any of the preceding compounds of Formula (II), wherein Y is O. In still another preferred embodiment, a compound of Formula (II) comprises any of the preceding compounds of Formula (II), wherein Y is NH.

In still another embodiment, a compound of Formula (II) comprises any of the preceding compounds of Formula (II), wherein n is an integer from 1-5. In a preferred embodiment, a compound of Formula (II) comprises any of the preceding compounds of Formula (II), wherein n is 2.

In another embodiment, provided herein are compounds comprising Formula (III) and salts (e.g., pharmaceutically acceptable salts) thereof:

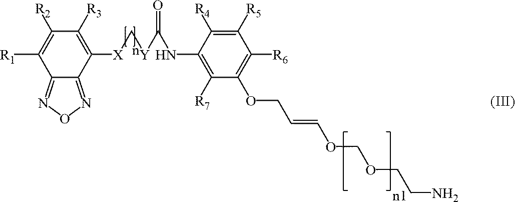

wherein X and Y are independently selected from O, S, NH, Se, (OC₂H₄)₁-₅,
wherein R₁, R₂, R₃, R₄, R₅, R₆, and R₇ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur;
n is an integer from 1-5; and
n1 is an integer from 1-5.

In an embodiment, a compound of Formula (III) comprises any of the preceding compounds of Formula (III), wherein R₁ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (II) comprises any of the preceding compounds of Formula (III), wherein R₁ is NO₂.

In another embodiment, a compound of Formula (III) comprises any of the preceding compounds of Formula (III), wherein R₂ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (III) comprises any of the preceding compounds of Formula (III), wherein R₂ is H.

In still another embodiment, a compound of Formula (III) comprises any of the preceding compounds of Formula (III), wherein R₃ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (III) comprises any of the preceding compounds of Formula (III), wherein R₃ is H.

In yet another embodiment, a compound of Formula (III) comprises any of the preceding compounds of Formula (III), wherein R₄ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (III) comprises any of the preceding compounds of Formula (III), wherein R₄ is H.

In still yet another embodiment, a compound of Formula (III) comprises any of the preceding compounds of Formula (III), wherein R₅ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (III) comprises any of the preceding compounds of Formula (III), wherein R₅ is H.

In another embodiment, a compound of Formula (III) comprises any of the preceding compounds of Formula (III), wherein R₆ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (III) comprises any of the preceding compounds of Formula (III), wherein R₆ is H.

In yet another embodiment, a compound of Formula (III) comprises any of the preceding compounds of Formula (III), wherein R₇ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (III) comprises any of the preceding compounds of Formula (III), wherein R₇ is H.

In still yet another embodiment, a compound of Formula (III) comprises any of the preceding compounds of Formula (III), wherein X is selected from the group O, S, NH, Se, (OC₂H₄)₁₋₅. In a preferred embodiment, a compound of Formula (III) comprises any of the preceding compounds of Formula (III), wherein X is S. In another preferred embodiment, a compound of Formula (III) comprises any of the preceding compounds of Formula (III), wherein X is O. In still another preferred embodiment, a compound of Formula (III) comprises any of the preceding compounds of Formula (III), wherein X is NH.

In another embodiment, a compound of Formula (III) comprises any of the preceding compounds of Formula (III), wherein Y is selected from the group O, S, NH, Se, (OC₂H₄)₁₋₅. In a preferred embodiment, a compound of Formula (III) comprises any of the preceding compounds of Formula (III), wherein Y is S. In another preferred embodiment, a compound of Formula (III) comprises any of the preceding compounds of Formula (III), wherein Y is O. In still another preferred embodiment, a compound of Formula (III) comprises any of the preceding compounds of Formula (III), wherein Y is NH.

In still another embodiment, a compound of Formula (III) comprises any of the preceding compounds of Formula (III), wherein n is an integer from 1-5. In a preferred embodiment, a compound of Formula (III) comprises any of the preceding compounds of Formula (III), wherein n is 2.

In still yet another embodiment, a compound of Formula (III) comprises any of the preceding compounds of Formula (III), wherein n1 is an integer from 1-5. In a preferred embodiment, a compound of Formula (III) comprises any of the preceding compounds of Formula (III), wherein n1 is 1.

In another embodiment, provided herein are compounds comprising Formula (IV) and salts (e.g., pharmaceutically acceptable salts) thereof:

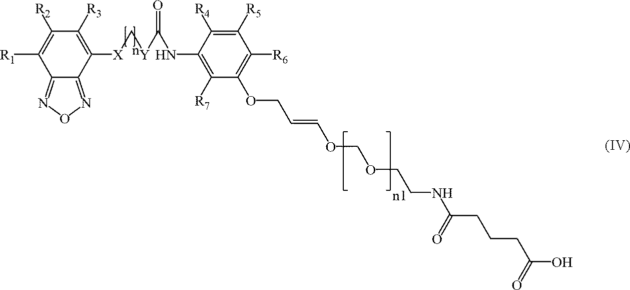

wherein X and Y are independently selected from O, S, NH, Se, (OC₂H₄)_{1–5},
wherein R₁, R₂, R₃, R₄, R₅, R₆, and R₇ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur;
n is an integer from 1-5; and
n1 is an integer from 1-5.

In an embodiment, a compound of Formula (IV) comprises any of the preceding compounds of Formula (IV), wherein R₁ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₁ is —NO₂.

In another embodiment, a compound of Formula (IV) comprises any of the preceding compounds of Formula (IV), wherein R₂ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₂ is H.

In still another embodiment, a compound of Formula (IV) comprises any of the preceding compounds of Formula (IV), wherein R₃ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₃ is H.

In yet another embodiment, a compound of Formula (IV) comprises any of the preceding compounds of Formula (IV), wherein R₄ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₄ is H.

In still yet another embodiment, a compound of Formula (IV) comprises any of the preceding compounds of Formula (IV), wherein R₅ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₅ is H.

In another embodiment, a compound of Formula (IV) comprises any of the preceding compounds of Formula (IV), wherein R₆ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₆ is H.

In yet another embodiment, a compound of Formula (IV) comprises any of the preceding compounds of Formula (IV), wherein R₇ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur. In a preferred embodiment, a compound of Formula (IV) comprises any of the preceding compounds of Formula (IV), wherein R₇ is H.

In still yet another embodiment, a compound of Formula (IV) comprises any of the preceding compounds of Formula (IV), wherein X is selected from O, S, NH, Se, (OC₂H₄)₁₋₅. In a preferred embodiment, a compound of Formula (IV) comprises any of the preceding compounds of Formula (IV), wherein X is S. In another preferred embodiment, a compound of Formula (IV) comprises any of the preceding compounds of Formula (IV), wherein X is O. In still another preferred embodiment, a compound of Formula (IV) comprises any of the preceding compounds of Formula (IV), wherein X is NH.

In another embodiment, a compound of Formula (IV) comprises any of the preceding compounds of Formula (IV), wherein Y is selected from O, S, NH, Se, (OC₂H₄)₁₋₅ In a preferred embodiment, a compound of Formula (IV) comprises any of the preceding compounds of Formula (IV), wherein Y is S. In another preferred embodiment, a compound of Formula (IV) comprises any of the preceding compounds of Formula (IV), wherein Y is O. In still another preferred embodiment, a compound of Formula (IV) comprises any of the preceding compounds of Formula (IV), wherein Y is NH.

In still another embodiment, a compound of Formula (IV) comprises any of the preceding compounds of Formula (IV), wherein n is an integer from 1-5. In a preferred embodiment, a compound of Formula (IV) comprises any of the preceding compounds of Formula (IV), wherein n is 2.

In still yet another embodiment, a compound of Formula (IV) comprises any of the preceding compounds of Formula (IV), wherein n1 is an integer from 1-5. In a preferred embodiment, a compound of Formula (IV) comprises any of the preceding compounds of Formula (IV), wherein n1 is 1.

It is understood that the present disclosure encompasses a composition comprising an isomer of compound of Formula (I), (II), (III), or (IV). The terms “isomer,” “isomeric form,” “stereochemically isomeric forms,” or “stereolsomeric forms,” as used herein, defines all possible isomeric as well as conformational forms, made up of the same atoms bonded by the same sequence of bonds but having different three-dimensional structures which are not interchangeable, which compounds or intermediates obtained during said process may possess. Unless otherwise mentioned or indicated, the chemical designation of a compound encompasses the mixture of all possible stereochemically isomeric forms which said compound may possess. Said mixture may contain all diastereoisomers, epimers, enantiomers, and/or conformers of the basic molecular structure of said compound. More in particular, stereogenic centers may have the R- or S-configuration, diastereoisomers may have a syn- or anti-configuration, substituents on bivalent cyclic saturated radicals may have either the cis- or trans-configuration and alkenyl radicals may have the E or Z-configuration. All stereochemically isomeric forms of said compound both in pure form or in admixture with each other are intended to be embraced within the scope of the present invention.

The compounds disclosed herein can exist as therapeutically acceptable salts. The present disclosure includes compounds listed above in the form of salts, including acid addition salts. Suitable salts include those: formed with both organic and inorganic acids. Such acid addition salts will normally be pharmaceutically acceptable. However, salts of non-pharmaceutically acceptable salts can be of utility in the preparation and purification of the compound in question. Basic addition salts can also be formed and be pharmaceutically acceptable. For more complete discussion of the preparation and selection of salts, refer to Pharmaceutical Salts: Properties, Selection, and Use (Stahl, P. Heinrich, Wiley VCHA, Zurich, Switzerland, 2002).

The term “therapeutically acceptable salt,” as used herein represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate aspartate, benzoate, benzenesttlfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansullonate (isethionate), lactate, maleate, malonate, DL-mandelate, mesitylenesulfonate, methanesulfonate, naphthylenesulfonate, nicotinate, 2-naplithalenesultanate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric,, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like. Non-limiting examples of pharmaceutically acceptable salts of the above compounds include, inorganic acid salts such as hydrochloric acid, hydrobromic acid, nitric acid, sulphuric acid, and also organic acids such as oxalic acid, formic acid, fumaric acid, maleic acid, tartaric acid, citric acid, succinic acid.

### (B) Detectable Label

In an aspect, a compound of Formula (I), (II), (III), or (IV) as discussed supra may be conjugated to a detectable label. A detectable label may be directly conjugated to a compound of the disclosure or may be indirectly conjugated to a compound of the disclosure. In an embodiment, a detectable label may be complexed with a chelating agent that is conjugated to a compound of the disclosure of the disclosure. In another embodiment, a detectable label may be complexed with a chelating agent that is conjugated to a linker that is conjugated to a compound of the disclosure of the disclosure. In still another embodiment, a detectable label may be conjugated to a linker that is conjugated to a compound of the disclosure of the disclosure. In still yet another embodiment, a detectable label may be directly attached to a compound of the disclosure. Single, dual or multiple labeling of a compound of the disclosure may be advantageous. For example, a compound of the disclosure may be conjugated to one, two, three, four, or five types of detectable labels.

As used herein, a “detectable label” is any type of label which, when attached to a compound of the disclosure renders the compound detectable. A detectable label may also be toxic to cells or cytotoxic. Accordingly, a detectable label may also be a therapeutic agent or cytotoxic agent. In general, detectable labels may include but are not limited to luminescent molecules, chemiluminescent molecules, fluorochromes, fluorophores (e.g., cyanine dyes), fluorescent quenching agents, colored molecules, radioisotopes, radionuclides, cintillants, massive labels such as a metal atom (for detection via mass changes), biotin, avidin, streptavidin, protein A, protein G, antibodies or fragments thereof, Grb2, polyhistidine, Ni²⁺, Flag tags, myc tags, heavy metals, enzymes, alkaline phosphatase, peroxidase, luciferase, electron donors/acceptors, acridinium esters, colorimetric substrates. The skilled artisan would readily recognize other useful labels that are not mentioned above, which may be employed in the operation of the present disclosure. Particularly useful detectable labels of the disclosure include but are not limited to copper-64, zirconium-89, fluorine-18, iodine-124, yttrium-86, yttrium-90, iodine-131, lutetium-177, bromine-76, biotin, LS288, LS606, LS1074, LS301, DOTA, DFO, DTPA, cross-bridged ligands,

A detectable label emits a signal that can be detected by a signal transducing machine. In some cases, the detectable label can emit a signal spontaneously, such as when the detectable label is a radionuclide. In other cases the detectable label emits a signal as a result of being stimulated by an external field such as when the detectable label is a relaxivity metal. Examples of signals include, without limitation, gamma rays, X-rays, visible light, infrared energy, and radiowaves. Examples of signal transducing machines include, without limitation, gamma cameras including SPECT/CT devices, PET scanners, fluorimeters, and Magnetic Resonance Imaging (MRI) machines. As such, the detectable label comprises a label that can be detected using magnetic resonance imaging, scintigraphic imaging, ultrasound, or fluorescence. In a specific embodiment, the detectable label comprises a label that can be detected using positron emission tomography, single photon emission computed tomography, gamma camera imaging, or rectilinear scanning.

Suitable fluorophores include, but are not limited to, fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes. A compound of the disclosoure can be labeled for fluorescence detection by labeling the agent with a fluorophore using techniques well known in the art (see, e.g., Lohse et al., Bioconj Chem 8:503-509 (1997)). For example, many known dyes are capable of being coupled to NH₂-terminal groups i.e. a compound of formula (III). In a specific embodiment, an alkyne modified dye, such an Alexa Fluor dye, may be clicked to a compound of the disclosure (e.g., a compound of formula (II)), for example, using click chemistry (Kolb et al., Angew Chem Int Ed 2001; 40: 2004-2021, which incorporated by reference in its entirety).

A radionuclide may be a γ-emitting radionuclide, Auger-emitting radionuclide, β-emitting radionuclide, an α-emitting radionuclide, or a positron-emitting radionuclide. A radionuclide may be a detectable label and/or a therapeutic agent. Non-limiting examples of suitable radionuclides may include carbon-11, nitrogen-13, oxygen-15, fluorine-18, fluorodeoxyglucose-18, phosphorous-32, scandium-47, copper-64, 65 and 67, gallium-67 and 68, bromine-75, 76, 77 and 80m, rubidium-82, strontium-89, zirconium-89, yttrium-86 and 90, ruthenium-95, 97,103 and 105, rhenium-99m, 101, 105, 186 and 188, technetium-99m, rhodium-105, mercury-107, palladium-109, indium-111, silver-111, indium-113m, lanthanide-114m, tin-117m, tellurium-121m, 122m and 125m, iodine-122, 123, 124, 125, 126, 131 and 133, praseodymium-142, promethium-149, samarium-153, gadolinium-159, thulium-165, 167 and 168, dysprosium-165, holmium-166, lutetium-177, rhenium-186 and 188, iridium-192, platinum-193 and 195m, gold-199, thallium-201, titanium-201, astatine-211, bismuth-212 and 213, lead-212, radium-223, actinium-225, and nitride or oxide forms derived there from.

A variety of metal atoms may be used as a detectable label. The metal atom may generally be selected from the group of metal atoms comprised of metals with an atomic number of twenty or greater. For instance, the metal atoms may be calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms. In some embodiments, the metal atoms may be selected from the group comprising alkali metals with an atomic number greater than twenty. In other embodiments, the metal atoms may be selected from the group comprising alkaline earth metals with an atomic number greater than twenty. In one embodiment, the metal atoms may be selected from the group of metals comprising the lanthanides. In another embodiment, the metal atoms may be selected from the group of metals comprising the actinides. In still another embodiment, the metal atoms may be selected from the group of metals comprising the transition metals. In yet another embodiment, the metal atoms may be selected from the group of metals comprising the poor metals. In other embodiments, the metal atoms may be selected from the group comprising gold atoms, bismuth atoms, tantalum atoms, and gadolinium atoms. In preferred embodiments, the metal atoms may be selected from the group comprising metals with an atomic number of 53 (i.e. iodine) to 83 (i.e. bismuth). In an alternative embodiment, the metal atoms may be atoms suitable for magnetic resonance imaging. In another alternative embodiment, the metal atoms may be selected from the group consisting of metals that have a K-edge in the x-ray energy band of CT. Preferred metal atoms include, but are not limited to, manganese, iron, gadolinium, gold, and iodine.

The metal atoms may be metal ions in the form of +1, +2, or +3 oxidation states. For instance, non-limiting examples include Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe⁺, F3⁺, Pb²⁺, Mn²⁺, Mn³⁺, Mn⁴⁺, Mn⁷⁺, Hg²⁺, Ni²⁺, Ni³⁺, Ag+, Sr²⁺, Sn²⁺, Sn⁴⁺, and Zn²⁺. The metal atoms may comprise a metal oxide. For instance, non-limiting examples of metal oxides may include iron oxide, manganese oxide, or gadolinium oxide. Additional examples may include magnetite, maghemite, or a combination thereof.

### (C) Components of the Composition

The present disclosure also provides pharmaceutical compositions. The pharmaceutical composition comprises a compound of Formula (I), (II), (III), or (IV), as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

### (I) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

### (II) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

### (III) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

### (IV) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

### (V) pH Modifier

In various embodiments, the excipient may be a pH modifier. By way of non-limiting example, the pH modifying agent may be sodium carbonate, sodium bicarbonate, sodium citrate, citric acid, or phosphoric acid.

### (VI) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

### (VII) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

### (VIII) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

### (IX) Lubricant

In a further embodiment, the excipient may be a lubricant. Non-limiting examples of suitable lubricants include minerals such as talc or silica; and fats such as vegetable stearin, magnesium stearate, or stearic acid.

### (X) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

### (XI) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

### (XII) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

### (D) Administration Forms

The agents and compositions described herein can be formulated by any conventional manner using one or more pharmaceutically acceptable carriers or excipients as described in, for example, Remington’s Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington’s Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The concentration of a compound of the present disclosure in the fluid pharmaceutical formulations can vary widely, i.e., from less than about 0.05% usually or at least about 2-10% to as much as 30 to 50% by weight and will be selected primarily by fluid volumes, viscosities, etc., in accordance with the particular mode of administration selected. For example, the concentration may be increased to lower the fluid load associated with treatment. The amount of pharmaceutical composition administered will depend upon the particular therapeutic entity entrapped inside the nanoparticle, the type of nanoparticle being used, and the judgment of the clinician. Generally the amount of pharmaceutical composition administered will be sufficient to deliver a therapeutically effective dose of the particular therapeutic entity.

The quantity of pharmaceutical composition necessary to deliver a therapeutically effective dose can be determined by routine in vitro and in vivo methods, common in the art of drug testing. See, for example, D. B. Budman, A. H. Calvert, E. K. Rowinsky (editors). Handbook of Anticancer Drug Development, LWW, 2003. Therapeutically effective dosages for various therapeutic entities are well known to those of skill in the art; and according to the present disclosure a therapeutic entity delivered via the pharmaceutical liposome composition of the present invention provides at least the same, or 2-fold, 4-fold, or 10-fold higher activity than the activity obtained by administering the same amount of the therapeutic entity in its routine non-liposome formulation. Typically the dosages for the pharmaceutical composition of the present disclosure range between about 0.005 and about 500 mg of the therapeutic entity per kilogram of body weight, most often, between about 0.1 and about 100 mg therapeutic entity/kg of body weight.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Additional formulations of pharmaceutical delivery systems may be in, for example, Hoover, John E., Remington’s Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington’s Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. A suitable pharmaceutically acceptable carrier to maintain optimum stability, shelf-life, efficacy, and function of the delivery system would be apparent to one of ordinary skill in the art.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington’s Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising a compound of Formula (I), (II), (III), or (IV) is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles (e.g., protein nanoparticles (NMs) like human serum albumin (HSA), Transferrin (Tf)), liposomes, microemulsions, micelles, dendrimers, and other phospholipid-containing systems. Methods of incorporating compositions into delivery vehicles are known in the art.

In some embodiments, the suitable vehicle used to encapsulate a compound of Formula (I), (II), (III), or (IV) is further labeled on its surface with one or more targeting ligands. Non-limiting examples of targeting ligands include targeting peptides, which may be natural or synthetic peptides, such as, for example, a targeting antibody or antibody fragments, targeting glycans (e.g., sugar molecules targeting cell surface receptors), nucleic acids (e.g., single stranded or double stranded DNA, various forms of RNA (e.g., siRNA, and the like), lipids, carboyhydrates (e.g., oligosaccharides, polysaccharides, sugars, and the like), perfluorocarbons, phosphonic acids and bis-phosphonic acids. Preferred targeting ligands include LLP2A or CS1 antibody targeting groups and bone homing agents like LS288, perfluorocarbons, phosphonic acids, bis-phosphonic acids.

In one alternative embodiment, a liposome delivery vehicle may be utilized. Liposomes, depending upon the embodiment, are suitable for delivery of a compound of Formula (I), (II), (III), or (IV) in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the compound of Formula (I), (II), (III), or (IV) may be selectively delivered to a cell by encapsulation in a liposome that fuses with the targeted cell’s membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a compound of Formula (I), (II), (III), or (IV) may be prepared by any known method of preparing liposomes for drug delivery, such as those described in the Examples below or as detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of the compound of Formula (I), (II), (III), or (IV), concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The compound of Formula (I), (II), (III), or (IV) may be encapsulated in a microemulsion by any method generally known in the art.

In yet another embodiment, a compound of Formula (I), (II), (III), or (IV) may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

In yet another embodiment, a compound of Formula (I), (II), (III), or (IV) may be delivered in a biopolymer-based nanoparticles, such as protein nanoparticles. Protein nanoparticles have been actively used as pharmaceutical and functional tools owing to their low toxicity and biodegradability, as exhibited throughout the many studies that have been conducted on the subject. Proteins exhibit unique functions and properties in biological materials and manufacturing fields and can be used as base materials for production of nanoparticles. Owing to their small size, protein nanoparticles can be transmitted through the cell via endocytosis. Protein nanoparticles have several advantages as a drug delivery system, such as biodegradability, stability, surface modification of particles, ease of particle size control, and they have less problems associated with toxicity issues, such as immunogenicity. Protein nanoparticles may be comprised of a variety of different types of proteins, including but not limited to fibroin, human serum albumin, gliadin, gelatin, legumin, 30Kc19, lipoprotein, ferritin and transferrin. Preferred protein nanoparticles include human serum albumin and transferrin nanoparticles. Method for producing protein nanoparticles are known in the art, for example, those described in Hong, Seyoung et al. “Protein-Based Nanoparticles as Drug Delivery Systems.” Pharmaceutics vol. 12,7 604. 29 Jun. 2020, doi:10.3390/pharmaceutics12070604, incorporated herein by reference in it’s entirety. The present disclosure is not particularly limited to a particular production method.

Dosages of a compound of Formula (I), (II), (III), or (IV) can vary between wide limits, depending upon the disease or disorder to be treated, the age and condition of the subject to be treated. In an embodiment where a composition comprising a compound of Formula (I), (II), (III), or (IV) is contacted with a sample, the concentration of a compound of Formula (I), (II), (III), or (IV) may be from about 1 µM to about 40 µM. Alternatively, the concentration of a compound of Formula (I), (II), (III), or (IV) may be from about 5 µM to about 25 µM. For example, the concentration of a compound of Formula (I), (II), (III), or (IV) may be about 1, about 2.5 about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, or about 40 µM. Additionally, the concentration of a compound of Formula (I), (II), (III), or (IV) may be greater than 40 µM. For example, the concentration of a compound of Formula (I), (II), (III), or (IV) may be about 40, about 45, about 50, about 55, about 60, about 65, about 70, about 75, about 80, about 85, about 90, about 95, or about 100 µM.

In an embodiment where the composition comprising a compound of Formula (I), (II), (III), or (IV) is administered to a subject, the dose of a compound of Formula (I), (II), (III), or (IV) may be from about 0.1 mg/kg to about 500 mg/kg. For example, the dose of a compound of Formula (I), (II), (III), or (IV) may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of a compound of Formula (I), (II), (III), or (IV) may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of a compound of Formula (I), (II), (III), or (IV) may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

As noted above, the agents or compositions described herein can also be used in combination with other therapeutic agents, as described further below. Thus, in addition to the therapies described herein, one may also provide to the subject other therapies known to be efficacious for treatment of the disease, disorder, or condition. In various examples, a method further comprises administering to the patient an additional cancer treatment. In some examples, the additional cancer treatment is chosen from the group comprising surgery, radiotherapy, chemotherapy, toxin therapy, immunotherapy, cryotherapy, gene therapy, and combinations thereof. In various examples, a chemotherapy agent is a drug or drug formulation. Non-limiting examples of drug formulations include polymeric micelle formulations, liposomal formulations, dendrimer formulations, polymer-based nanoparticle formulations, silica-based nanoparticle formulations, nanoscale coordination polymer formulations, nanoscale metal-organic framework formulations, inorganic nanoparticle formulations, and the like.

Various chemotherapy agents (e.g., chemotherapy drugs) can be used. Any FDA approved chemotherapy agent (e.g., chemotherapy drugs) can be used. Combinations of chemotherapy agents may be used.

In some embodiments, the additional drug or therapeutically active agent may be a genotoxic agent (e.g., a DNA-damaging agent or drug). As used herein “genotoxic therapy” refers to a treat of a tumor or cancer which utilizes the destructive properties of the treatment to induce DNA damage into tumor or cancer cells. The treatment is traditionally part of standardized regime. Any damage done to a tumor cancer is passed on to descendent cancer cells as proliferation continues. If this damage is severe enough, it will induce cells to undergo apoptosis. In non-limiting examples, a genotoxic therapy may include γ-irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, mitoxantrone, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), and estramustine.

In some embodiments, the additional active agent is bortezomib (BTZ). As described herein, Applicant has surprisingly discovered administration of a compound of the present disclosure synergized with BTZ in combination therapy to inhibit cancer cell growth.

Treatment in accord with the methods described herein can be performed prior to, concurrent with, or after conventional treatment modalities for a cancer or tumor.

Dosages of an additional drug or therapeutically active agent can vary between wide limits, depending upon the disease or disorder to be treated, the age and condition of the subject to be treated. In an embodiment where the composition further comprising at least one additional drug or therapeutically active agent is contacted with a sample, the concentration of the at least one additional drug or therapeutically active agent may be from about 0.01 µM to about 10 µM. Alternatively, the concentration of the at least one additional drug or therapeutically active agent may be from about 0.01 µM to about 5 µM. For example, the concentration of the at least one additional drug or therapeutically active agent may be about 0.01, about 0.05, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, or about 10 µM. Additionally, the concentration of the at least one additional drug or therapeutically active agent be greater than 10 µM. For example, the concentration of the at least one additional agent may be about 10, about 15, about 20, about 25, about 30, about 35, about 40, about 45, about 50, about 55, about 60, about 65, about 70, about 75, about 80, about 85, about 90, about 95, or about 100 µM.

In an embodiment where the composition further comprising at least one additional drug or therapeutically active agent administered to a subject, the dose of the additional drug or therapeutically active agent may be from about 0.1 mg/kg to about 500 mg/kg. For example, the dose of the least one additional drug or therapeutically active agent may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the least one additional drug or therapeutically active agent may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of the least one additional drug or therapeutically active agent may be about 300 mg/kg, about 325 mg/kg, about 350 mg/kg, about 375 mg/kg, about 400 mg/kg, about 425 mg/kg, about 450 mg/kg, about 475 mg/kg, or about 500 mg/kg.

Generally, a safe and effective amount of a composition is administered, for example, that amount that would cause the desired therapeutic effect in a subject while minimizing undesired side effects. In various embodiments, an effective amount of a composition described herein can substantially reduce the growth or spread of cancer in a subject. In some embodiments, an effective amount is an amount capable of treating a cancer or tumor. In some embodiments, an effective amount is an amount capable of treating one or more symptoms associated with a cancer or tumor.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a composition as disclosed herein can occur as a single event or over a time course of treatment. For example, one or more of a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A dosing schedule of, for example, once/week, twice/week, three times/week, four times/week, five times/week, six times/week, seven times/week, once every two weeks, once every three weeks, once every four weeks, once every five weeks, and the like, is available for the disclosure. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

As one aspect of the present disclosure contemplates the treatment of the disease/conditions with the compounds of the disclosure, the disclosure further relates to pharmaceutical compositions in kit form. When the composition of the disclosure is a part of a combination therapy with a secondary therapeutic agent, the kit may comprise two separate pharmaceutical compositions: one of compound of the present disclosure, and another of a second therapeutic agent. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the invention, the subject may be a human or any other animal.

### II. Methods

The present disclosure relates, in general, to methods of preparing compounds of the disclosure and to methods of using the compounds and/or pharmaceutical compositions as described herein.

### (A) Methods of Preparation

Many general references providing commonly known chemical synthetic schemes and conditions useful for synthesizing the disclosed compounds are available (see, e.g., Smith and March, March’s Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/I, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of structural formula (I), (II), (III) or (IV) can be prepared according to Schemes 1-5, general procedures (below), and/or analogous synthetic procedures. One of skill in the art can adapt the reaction sequences of Schemes 1-5, general procedures, and Examples to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### (I) General Procedures

Representative synthetic procedures for the preparation of compounds of the invention are outlined below in Schemes 1-5.

[symbol]

[symbol]

[symbol]

[symbol]

[symbol]

### (B) Therapeutic Methods

The present disclosure provides compounds and pharmaceutical compositions comprising the same which are useful for treating a cancer or tumor in a subject. In some embodiments, administration of a composition of the present disclosure results in ERK1/2 activation to induce caspase 3/7-dependent apoptosis in a cancer or tumor cell. In some embodiments, administration of a composition of the present disclosure results in increased expression of pro-apoptotic protein Bax in a cancer or tumor cell. Thus, in some embodiments, the methods of the present disclosure are useful for reducing tumor or cancer progression or prolonging the survival of subject having a cancer or tumor. In another aspect, the disclosure provides a composition as disclosed herein for use in vitro, in vivo, or ex vivo. Suitable compositions for use in the methods of the present disclosure are disclosed herein, for instance those described in Section I.

Therefore, in one aspect, the methods and compositions comprising them, can be administered to an individual to kill endogenous tissue or cells. The tissue can be undesirable tissue that has arisen due to transformation, such as a tumor, cancer. As used herein, the term “cancer” includes a wide variety of malignant neoplasms. These can be caused by viral infection, naturally occurring transformation, or exposure to environmental agents.

In some examples, the methods can be useful for treating a cancer or tumor in a subject. The term “treating a cancer or tumor” includes, but is not limited to, preventing or reducing the development of a cancer or tumor, reducing the symptoms of cancer or tumor, suppressing or inhibiting the growth of an established cancer or tumor, preventing metastasis and/or invasion of an existing cancer or tumor, promoting or inducing regression of the cancer or tumor, inhibiting or suppressing the proliferation of cancerous or tumor cells, reducing angiogenesis or increasing the amount of apoptotic cancer or tumor cells, thereby treating cancer or a tumor.

Non-limiting examples of cancers or tumors that may be treated with a method of the disclosure may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing’s sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood).

In one embodiment, this method generally includes the steps of providing at least one nanoparticle composition as described herein; administering the nanoparticle composition to a subject or cell and administering an excitation source such that the nanoparticle emits electromagnetic radiation having a first wavelength when irradiated with the excitation source having electromagnetic radiation having a second wavelength (e.g. visible light, near-infrared light, and X-ray). In this embodiment, a photosensitizer attached to the nanoparticle absorbs the electromagnetic radiation having a first wavelength thereby providing the photodynamic therapy. The term “photosensitizer” (PS) refers to a chemical compound or moiety that can be excited by light of a particular wavelength, typically visible or near-infrared (NIR) light, and produce a reactive oxygen species (ROS). For example, in its excited state, the photosensitizer can undergo intersystem crossing and transfer energy to oxygen (O2) (e.g., in tissues being treated by PDT) to produce ROSs, such as singlet oxygen (102). Any known type of a photosensitizer can be used in accordance with the presently disclosed subject matter. In some embodiments, the photosensitizer is a porphyrin, a chlorophyll, a dye, or a derivative or analog thereof. In some embodiments, phophyrins, chlorins, bacteriochlorins, or porphycenes can be used. In some embodiments, the photosensitizer can have one or more functional groups, such as carboxylic acid, amine, or isothiocyanate, e.g., for using in attaching the photosensitizer to another molecule or moiety, such as an organic bridging ligand or a SBU, and/or for providing an additional site or sites to enhance coordination or to coordinate an additional metal or metals. In some embodiments, the photosensitizer is a porphyrin or a derivative or analog thereof. Exemplary porphyrins include, but are not limited to, hematoporphyrin, protoporphyrin and tetraphenylporphyrin (TPP). Exemplary porphyrin derivatives include, but are not limited to, pyropheophorbides, bacteriochlorophylls, chlorophyll a, benzoporphyrin derivatives, tetrahydroxyphenyl chlorins, purpurins, benzochlorins, naphthochlorins, verdins, rhodins, oxochlorins, azachlorins, bacteriochlorins, tolyporphyrins and benzobacteriochlorins. Porphyrin analogs include, but are not limited to, expanded porphyrin family members (such as texaphyrins, sapphyrins and hexaphyrins), porphyrin isomers (such as porphycenes, inverted porphyrins, phthalocyanines, and naphthalocyanines), and TPP substituted with one or more functional groups.

In some embodiments, the present disclosure provides a methods for treating tumors or cancer cells which are resistant to apoptosis. In some embodiments, the disclosure provides a method of treating a cancer or tumor by administering a composition of the disclosure sequentially or simultaneously with another cancer therapy. When administered sequentially the additional agent may be administered before or after the compound of the disclosure. In a preferred embodiment, the additional agent is bortezomib.

In some embodiments, a compounds of the disclosure is administrated as a photosensitizer for use with photodynamic therapy or Cerenkov induced therapy in a method to treat a cancer or tumor. A photosensitizer is generally a macrocyclic organic complex, which absorbs radiation in the range of from about 300 nm to about 900 nm, typically from about 400 nm to about 800 nm, and are capable of transferring their absorbed energy to molecular oxygen to generate singlet oxygen. Thus, the present disclosure provides a method of treating a cancer or tumor cell, the method comprising contacting the cancer or tumor cell with an effective amount of a compound of the disclosure and an excitation source. In various embodiments, contact with the disclosed compounds and excitation source results in energy transfer to the photosensitizers and the subsequent generation of singlet oxygen which are needed for effective cancer treatment.

The term “effective amount”, as used herein, means an amount that leads to measurable effect, e.g., cancer cell death. The effective amount may be determined by using the methods known in the art and/or described in further detail in the examples.

In another aspect, the disclosure provides for a method of imaging or detecting a tumor or a cancer in a subject, the method generally comprising administering to the subject a composition comprising a compound of the present disclosure conjugated to a detectable label, and imaging the cancer or tumor in the subject.

In other aspects, compositions of the disclosure may be delivered to a cancer cell in vitro. A cancer cell may be a cancer cell line cultured in vitro. In some alternatives of the embodiments, a cancer cell line may be a primary cell line that is not yet described. Methods of preparing a primary cancer cell line utilize standard techniques known to individuals skilled in the art. In other alternatives, a cancer cell line may be an established cancer cell line. A cancer cell line may be adherent or non-adherent, or a cell line may be grown under conditions that encourage adherent, non-adherent or organotypic growth using standard techniques known to individuals skilled in the art. A cancer cell line may be contact inhibited or non-contact inhibited.

In some embodiments, the cancer cell line may be an established human cell line derived from a tumor. Non-limiting examples of cancer cell lines derived from a tumor may include the MM cell lines MM.1S, H929, and RPMI, osteosarcoma cell lines 143B, CAL-72, G-292, HOS, KHOS, MG-63, Saos-2, or U-2 OS; the prostate cancer cell lines DU145, PC3 or Lncap; the breast cancer cell lines MCF-7, MDA-MB-438 or T47D; the myeloid leukemia cell line THP-1, the glioblastoma cell line U87; the neuroblastoma cell line SHSY5Y; the bone cancer cell line Saos-2; the colon cancer cell lines WiDr, COLO 320DM, HT29, DLD-1, COLO 205, COLO 201, HCT-15, SW620, LoVo, SW403, SW403, SW1116, SW1463, SW837, SW948, SW1417, GPC-16, HCT-8, HCT 116, NCI-H716, NCI-H747, NCI-H508, NCI-H498, COLO 320HSR, SNU-C2A, LS 180, LS 174T, MOLT-4, LS513, LS1034, LS411N, Hs 675.T, CO 88BV59-1, Co88BV59H21-2, Co88BV59H21-2V67-66, 1116-NS-19-9, TA 99, AS 33, TS 106, Caco-2, HT-29, SK-CO-1, SNU-C2B or SW480; B16-F10, RAW264.7, the F8 cell line, or the pancreatic carcinoma cell line Panc1. In an exemplary embodiment, a method of the disclosure may be used to contact a cell of a MM cell line.

Generally, the methods as described herein comprise administration of a therapeutically effective amount of a nanoparticle composition of the disclosure to a subject. The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Procedure, Methods and Characterization of LS1090 Analogues

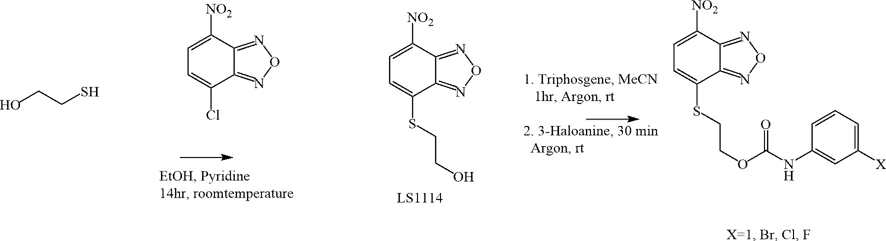

To a solution of 4-chloro-7-nitrobenzofurazan (1 gm, 5 mmol) and 2-mercaptoethanol (0.54 ml, 7.7 mmol) in ethanol (15 mL) at 0° C., pyridine (1.1 mL, 7.7 mmol) was added dropwise under nitrogen and the solution was allowed to stir overnight while allowing the reaction mixture to attain the room temperature. Upon completion of reaction, indicated by the disappearance of starting material by thin layer chromatography (TLC), the solution was removed under reduced pressure. The compound was extracted with ethyl acetate, washed with saturated solution of sodium bicarbonate and brine solution and dried with sodium sulfate. The organic solvent was removed under reduced pressure and purified by flash column chromatography using eluent hexane: ethyl acetate from 10:1 to 1:1. Desired organic fragments were collected and removed under pressure to obtain brownish yellow colored compound LS1114.

To a solution of triphosgene (85 mg, 0.28 mmol) in acetonitrile (5 mL), a solution of LS1114 (56 mg, 0.23 mmol) and triethylamine (50 µL, 0.37 mmol) was added in a dropwise manner. The solution was stirred under argon for 1 hour. Then the solution of 3-haloaniline (0.23 mmol) in acetonitrile (0.5 mL) was added and stirred for 30 minutes. Upon completion of reaction indicated by the disappearance of starting material, water was added and solution was concentrated under reduced pressure. The compound was extracted with ethyl acetate, dried over Na₂SO₄, and removed under reduced pressure. The crude product was purified by flash column chromatography with eluent hexane: ethyl acetate containing 1% NEt₃. Desired fragment were collected and removed under reduced pressure to obtain yellow product.

### Characterization

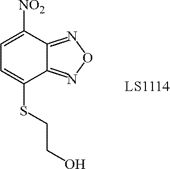 [SMILES: O=[N+]([O-])c1ccc(SCCO)c2nonc12]

2-(nitrobenzo[c][1,2,5]oxadiazol-4-yl)thio)ethan-1-ol: ¹H NMR (400 MHz, d-DMSO): δ=8.53-8.51 (d, J=8, 1H), 7.52-7.50 (d, J=8.0 Hz, 1H), 5.21 (br, 1H), 3.79-3.85 (t, J=4.0 Hz, 2H), 3.45-3.42 ppm (t, J=4.0 Hz, 2H); ¹³C NMR (400 MHz, d-DMSO): δ =150.04, 143.45, 141.23, 133.20, 132.88, 123.02, 59.63, 35.00 ppm.

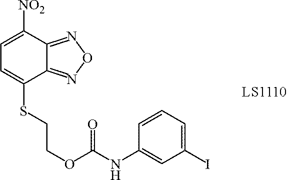 [SMILES: O=C(Nc1cccc(I)c1)OCCSc1ccc([N+](=O)[O-])c2nonc12]

2-((7-nitrobenzo[c][1,2,5]oxadiazol-4-yl)thio)ethyl (3-iodophenyl)carbamate: ¹H NMR (400 MHz, d-DMSO): δ=9.83 (s, 1H), 8.57-8.55 (d, J=8.0 Hz, 1H), 7.87 (s, 1H), 7.66-7.64 (d, J=8.0 Hz, 1H), 7.41-7.39 (d, J=8.0 Hz, 1H), 7.35-7.33 (d, J=8.0 Hz, 1H), 7.08-7.04 (t, J=8.0 Hz, 1H), 4.48-4.45 (d, J=5.2 Hz, 2H), 3.74-3.72 (t, J=5.6 Hz, 2H); ¹³C NMR (400 MHz, d-DMSO): δ =153.81, 150.19, 143.61, 141.20, 139.53, 133.46, 133.16, 132.08, 131.69, 127.28, 124.00, 118.47, 95.59, 62.61, 31.09 ppm.

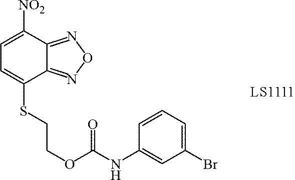 [SMILES: O=C(Nc1cccc(Br)c1)OCCSc1ccc([N+](=O)[O-])c2nonc12]

2-(nitrobenzo[c][1,2,5]oxadiazol-4-yl)thio)ethyl (3-bromophenyl)carbamate: ¹H NMR (400 MHz, d-DMSO): δ=9.91 (s, 1H), 8.56-8.54 (d, J=8.0 Hz, 1H), 7.70 (s, 1H), 7.66-7.64 (d, J=8.0 Hz, 1H), 7.39-7.37 (d, J=8.0 Hz, 1H), 7.24-7.20 (t, J=8.0 Hz, 1H), 7.17-7.16 (d, J=8.0 Hz, 1H), 4.49-4.46 (t, J=5.6 Hz, 2H), 3.75-3.73 (t, J=5.6 Hz, 2H); ¹³C NMR (400 MHz, d-DMSO): δ =153.83, 150.15, 143.56, 141.40, 139.53, 133.42, 133.11, 131.61, 126.10, 123.88, 122.56, 121.38, 117.97, 62.62, 31.07 ppm.

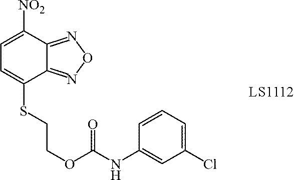 [SMILES: O=C(Nc1cccc(Cl)c1)OCCSc1ccc([N+](=O)[O-])c2nonc12]

2-(nitrobenzo[c][1,2,5]oxadiazol-4-yl)thio)ethyl (3-chlorophenyl)carbamate: ¹H NMR (400 MHz, d-DMSO): δ=9.92 (s, 1H), 8.56-8.54 (d, J=8.0 Hz, 1H), 7.65-7.63 (d, J=8.0 Hz, 1H), 7.54 (s, 1H), 7.39-7.27 (m, 2H), 7.05-7.03 (t, J=8.0 Hz, 1H), 4.49-4.46 (t, J=5.6 Hz, 2H), 3.75-3.72 (t, J=5.6 Hz, 2H); ¹³C NMR (400 MHz, d-DMSO): δ =153.97, 150.26, 143.66, 141.32, 139.61, 134.17, 133.52, 133.24, 131.45, 124.01, 123.33, 118.63, 117.70, 62.72, 31.18 ppm.

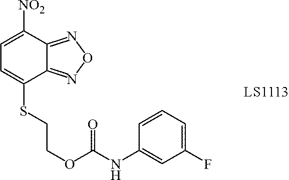 [SMILES: O=C(Nc1cccc(F)c1)OCCSc1ccc([N+](=O)[O-])c2nonc12]

2-(nitrobenzo[c][1,2,5]oxadiazol-4-yl)thio)ethyl (3-fluorophenyl)carbamate: ¹H NMR (400 MHz, d-DMSO): δ=9.96 (s, 1H), 8.58-8.56 (d, J=8.0 Hz, 1H), 7.68-7.65 (d, J=8.0 Hz, 1H), 7.36-7.27 (m, 2H), 7.20-7.18 (d, J=8.0 Hz, 1H), 6.84-6.80 (t, J= 8.4 Hz, 1H), 4.50-4.47 (t, J=5.6 Hz, 2H), 3.77-3.74 (t, J=5.6 Hz, 2H); ¹³C NMR (400 MHz, d-DMSO): δ =153.39, 149.70, 143.11, 141.06, 139.04, 132.99, 132.97, 130.88, 130.79, 123.47, 114.49, 109.56, 109.35, 62.11, 30.61 ppm.

### Example 2: Formulation of LS1090 and Analogues in Human Serum albumin (HSA) and Transferrin as HSA-LS1090/Analogue and Tf-LS1090/Analogue Nanoparticle (NPs) Respectively and Liposome Formulation

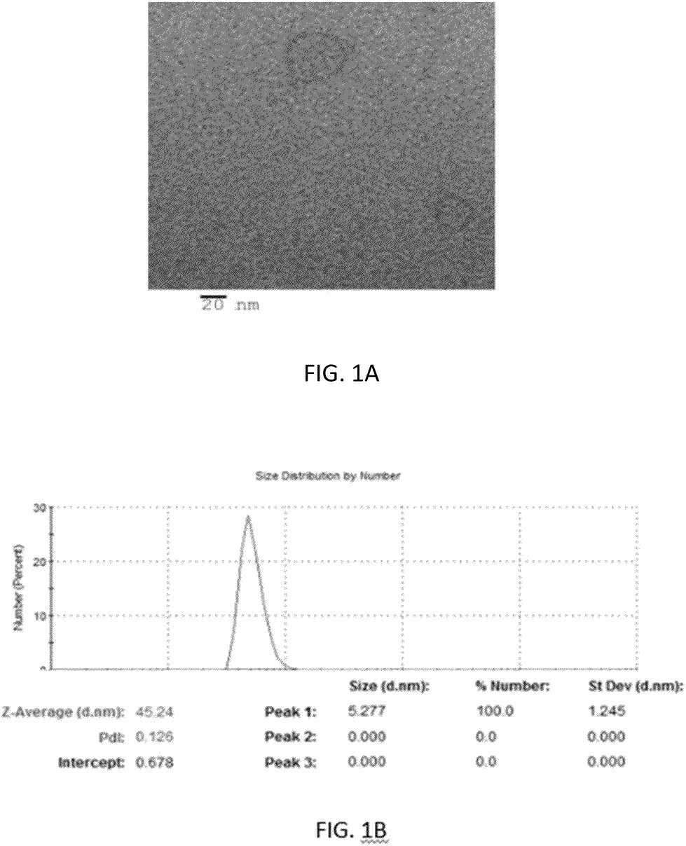

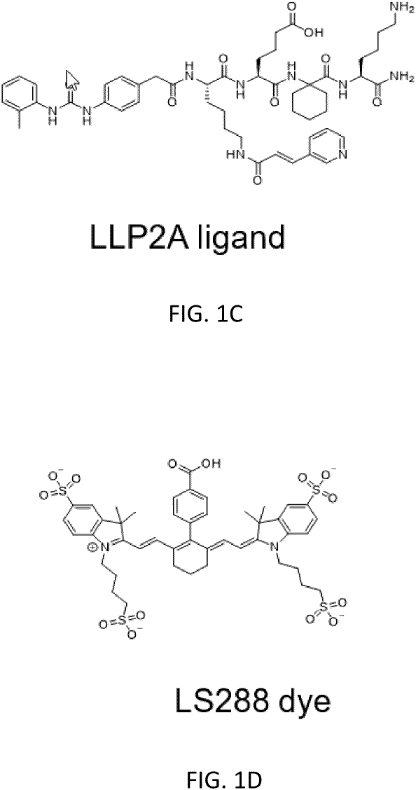

Rapidly proliferative cells are known to actively internalize albumin as a source of nitrogen, hence can act as a good drug delivery vehicle to the tumor cells. Earlier, it was demonstrated that Titanocene dichloride can be loaded in human serum albumin (HAS) nanoparticles and delivered to tumor cells in mice model. A well optimized method was used to formulate LS1090 successfully in to HSA nanoparticle (NP). Briefly, 100 mg of LS1090 or analogue in DMSO solution was shaken for 6 hours in 20 mL water containing 2.5 mg of HSA in a way that the total DMSO concentration be 0.1% in the resulting solution. The mixture was then purified through size exclusion chromatography to eliminate any unbound LS1090 followed by lyophilization to obtain the HSA-LS1090 NPs. The average hydrodynamic diameter of the particles were 5.24±1.25 nm indicated by dynamic light scattering (DLS) measurements by number distribution with polydispersity index (PDI) of 0.126. About 1% of the particles formed larger aggregates of diameter approximately 50-100 nm that created a bimodal distribution and skewed the z-average (45.25) (FIG. 1B). Tf-LS1090 was made following the exact same method that of HSA-LS1090 with same protein and LS1090 concentrations.

### Liposome Formulation of LS1090 and Its Analogues:

Components of the liposome: Different phosphocholine class lipids, pegylated derivatives of phosphatidylcholine class lipids and cholesterol was used in different ratios in forming the main core of the liposome. The different phosphocholine class lipids that were used:
1,2-dipalmitoyl-sn-glycero-3-phosphocholine (DPPC).
1,2-Didodecanoyl-sn-glycero-3-phosphocholine (DLPC).
1,2-dimyristoyl-sn-glycero-3-phosphocholine (DMPC).
1,2-distearoyl-sn-glycero-3-phosphocholine (DSPC).
1,2-dioleoyl-sn-glycero-3-phosphocholine (DOPC).
1,2-dimyristoyl-sn-glycero-3-phosphoethanolamine (DMPE).
1,2-Dipalmitoyl-sn-glycero-3-phosphoethanolamine (DPPE).
1,2-dioleoyl-sn-glycero-3-phosphoethanolamine (DOPE)
1,2-dimyristoyl-sn-glycero-3-phosphate (DMPA).
1,2-Dipalmitoyl-sn-glycero-3-phosphatidic acid (DPPA).
1,2-dioleoyl-sn-glycero-3-phosphate.
The different Pegylated Phosphocholine class lipids used are:
1,2-distearoyl-sn-glycero-3-phosphoethanolamine-N-[amino(polyethylene glycol)-5000] [DSPE-Peg (5000) Amine].
1,2-distearoyl-sn-glycero-3-phosphoethanolamine-N-[amino(polyethylene glycol)-2000] [DSPE-Peg (2000) Amine].
1,2-distearoyl-sn-glycero-3-phosphoethanolamine-N-[carboxy(polyethylene glycol)-5000] [DSPE-Peg (5000) carboxylic acid].
1,2-distearoyl-sn-glycero-3-phosphoethanolamine-N-[carboxy(polyethylene glycol)-2000] [DSPE-Peg (2000) carboxylic acid].
1,2-distearoyl-sn-glycero-3-phosphoethanolamine-N-[succinyl(polyethylene glycol)-5000] [DSPE-Peg (5000) succinyl].
1,2-distearoyl-sn-glycero-3-phosphoethanolamine-N-[succinyl(polyethylene glycol)-2000] [DSPE-Peg (2000) succinyl].
1,2-distearoyl-sn-glycero-3-phosphoethanolamine-N-[maleimide(polyethylene glycol)-5000] [DSPE-Peg (5000) maleimide].

Liposomes were prepared by extrusion method in total lipid concentration of 30 mM. Different lipid fractions in liposome: Phosphocholine 67.5%, Pegylated Phosphocholine 2.5%, Cholesterol 30%.

General preparation method: General preparation is described here using DPPC, DSPE-Peg (5000) Amine and cholesterol as components of liposome. Lipid stock solutions were prepared by dissolving each lipid in chloroform in different glass vials [Note: For any lipid component that are not soluble in chloroform, ethanol was used to prepare stock solutions for every lipid component]. The aliquots from the stock solutions were mixed in a different glass vial to give total 2 mL of solution with 30 mM lipid concentration. The solvent from the lipid mixture was evaporated off with a stream of nitrogen flow to obtain a lipid film. The lipid film was further dried under vacuum at -100 kPa for 2 h. The lipid film was then hydrated with 2 ml 120 mM ammonium sulfate. The lipid suspension thus formed was shaken at 60° C. for 30 minutes followed by freeze-though cycles. The liposomes were finally prepared from this suspension by extrusion through 50 nm polycarbonate membrane. The blank liposomes are typically below 40 nm in size (FIG. 1A) 0.005 mmol% of LS1090 or analogue to the lipid was dissolved in 10 µL dimethyl sulfoxide (DMSO) and added to the 2 mL lipid mixture. For targeting groups attachment, NHS ester of pegylated phosphocholine were used. LLp2A (FIG. 1C) or CS1 antibody was installed on the blank liposome by acid-amine coupling method prior to LS1090 loading. Bone homing LS288 (FIG. 1D) or phosphonic acid (0.001 mmol% to the lipid) in ammonium sulfate buffer was added to the liposome at the time of LS1090/ analogue addition when required. The liposomes were purified through size exclusion chromatography to remove any unbound small molecule and the buffer was exchanged into HEPES.

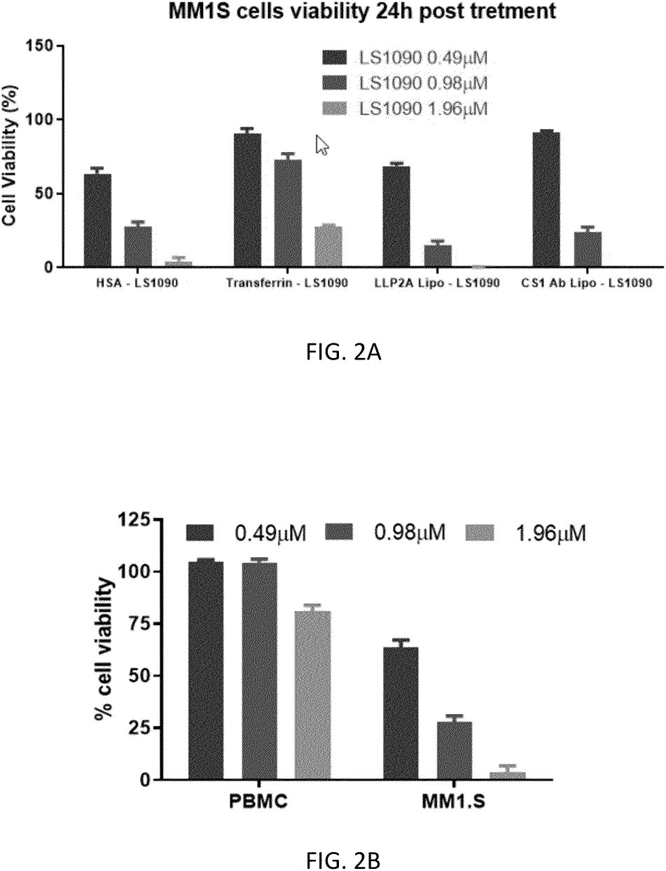

Nanoparticle formulation retains therapeutic effect and increases specificity of LS1090: The specificity and anti-MM activity of HSA-LS1090, was assessed in peripheral blood mononuclear cell (PBMC) viability after 24 hours incubation with the compound at the IC₅₀ for MM1.S concentration (0.49 µM). While LS1090 had significant toxic effect on PBMCs, HSA-LS1090 minimalized non-specific toxicity of LS1090. The HSA NP formulation did not reduce the toxicity of LS1090 on MM1.S cells, however, the toxic effect on PBMC was drastically reduced (FIG. 2).

HSA-LS1090 inhibits MM proliferation in vivo: In order to evaluate the anti-MM effect of HSA-LS1090 in vivo, a therapeutic effect in a disseminated medullar MM xenograft was tested. MM1.S cells stably transfected with GFP-firefly Luciferase (MM1.S-GFP-Luc), when administered intravenously (IV) in Fox Chase SCID beige mice, home to the bone marrow (BM) and are readily detectable by bioluminescence imaging (BLI) after 15-21 days from inoculation. On the third week from tumor inoculation, detecting the presence of the MM lesions in BM in mice with BLI photon flux of 106, we initiated therapy. Lyophilized HSA-LS1090 was reconstituted in 0.9% saline and 100 µL was administered IV per 20 g mouse (0.05 mg of effective LS-1090 concentration in HSA-LS1090 per 20 g mouse).

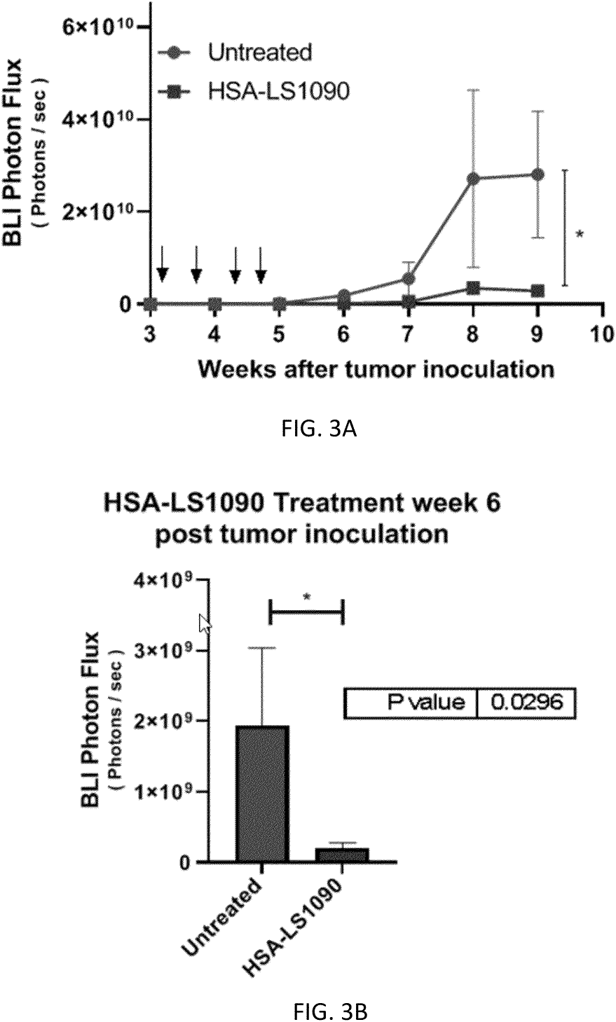

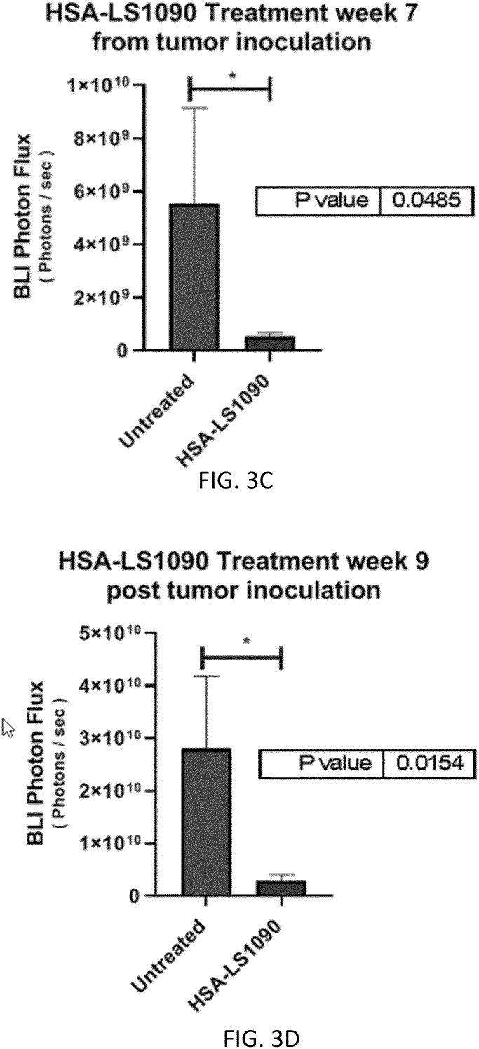

The concentration of LS1090 in the reconstituted HSA-LS1090 was confirmed by UV-VIS analysis prior to injection. The mice were subjected to four dosages of therapy in two days apart over two weeks. The therapy were stopped after four dosages and the tumor progression over time was monitored by in vivo BLI once a week. The mice with the Although the therapy was stopped at the end of week 5 post inoculation, the tumor progression in the treatment group was significantly lower compared to the untreated control until the end of week 9 (FIG. 3). Paralysis or loss of 15% of the original weight were taken as criteria of end point of the mice. The mice from the untreated group did not survive after week 9post inoculation, hence the comparative observation was stopped after week 9. The treated mice were visibly healthy with no weight loss until observed (14 weeks).

### Example 3: Pro-Apoptotic Small Molecule Inhibits Multiple Myeloma Progression Via ERK1/2 Signaling Pathway

Despite recent advances in multiple myeloma (MM) treatment, most patients experience frequent relapse, heightening the need for alternative chemotherapeutics. Using an unbiased cellular high throughput screening assay, we identified a small molecule, LS1090 that triggers caspase 3/7 dependent apoptosis in MM cells. LS1090 maintained its anti-MM cell activity in the presence of chemoresistance-enhancing growth factors, IL-6 and IGF-1. Isobologram analysis showed that LS1090 synergistically improved Bortezomib (BTZ) efficacy without inhibiting chymotrypsin-like activity of proteasomes. In disseminated human MM mouse model, LS1090 inhibited tumor growth and prolonged survival. These results highlight a viable strategy to augment therapeutic response of MM with minimal side effects.

Multiple myeloma (MM) is the second most frequent hematological disease characterized by plasma cell proliferation in the bone marrow (BM). The standard of care for MM includes a combination of immunomodulatory drugs (thalidomide, lenalidomide, pomalidomide), corticosteroids (dexamethasone), proteasome inhibitors (bortezomib (BTZ), carfilzomib, ixazomib), deacetylase inhibitors (panobinostat), and antibodies (elotuzumab, daratumumab).A major limiting factor of these drugs is the serious side effects, which include neurotoxicity, thrombocytopenia, peripheral neuropathy, and infection. Recent use of chimeric antigen receptor T cell therapy, which primes a patient’s T cells to recognize epitopes on malignant cells, showed early promises of recovery for few MM patients, but the disease relapses within a year of treatment. Also, the prohibitive cost, safety concerns, and neurotoxicity limit the broader clinical use of this therapy. Despite the significant improvements in survival afforded by these therapies, MM undergoes a vicious cycle of remission and relapse that eventually culminates in death.

Part of the challenge stems from the robust interactions between MM and the BM stromal cells to promote osteolysis, tumor growth, survival, immune suppression, and therapy resistance. In particular, these interactions also induce the secretion of cytokines and growth factors such as Il-6 and IGF-1 that promote the expression of anti-apoptotic proteins. Frontline therapies such as BTZ attempt to overcome resistance by targeting multiple facets of the MM survival pathways, including proteasome inhibition and enhancement of osteocyte viability. However, previous studies have demonstrated that MM circumvents proteasome inhibitor-induced apoptosis through a number of mechanisms that include increased proteasome activity and activation of anti-apoptotic proteins. These data suggest that new drugs that exert therapeutic effects via proteasome-independent pathways under conditions that support chemoresistance would complement existing standard of care therapies to minimize or prevent the frequency of MM relapse.

Here, the present Example identified a compound, 2-((7-nitrobenzo[c][1,2,5]oxadiazol-4-yl)thio)ethyl (3-(trifluoromethyl)phenyl)carbamate (LS1090) from a library of 21,400 small molecules, which selectively inhibited MM cell proliferation. Mechanistic studies showed that LS1090 triggers ERK1/2 activation to induce caspase 3/7-dependent apoptosis in the presence or absence of IL-6 and IGF-1. It was found that LS1090 exerts its biological effect via BTZ-independent mechanisms and synergized with BTZ in combination therapy to inhibit MM cell growth. Formulation of LS1090 in human serum albumin (HSA-LS1090) retained its selective cytotoxic effects on MM cells without harming normal peripheral blood mononuclear cells (PBMCs) cells in vitro. Extension of the pointing toward its ability to avoid systemic toxicity in vivo. Treatment of MM tumor-bearing mice with HSA-LS1090 inhibited tumor progression and prolonged survival. This work uncovers a new drug candidate that uses an alternative therapeutic pathway to BTZ, offering a new treatment paradigm for MM.

### Results and Discussion

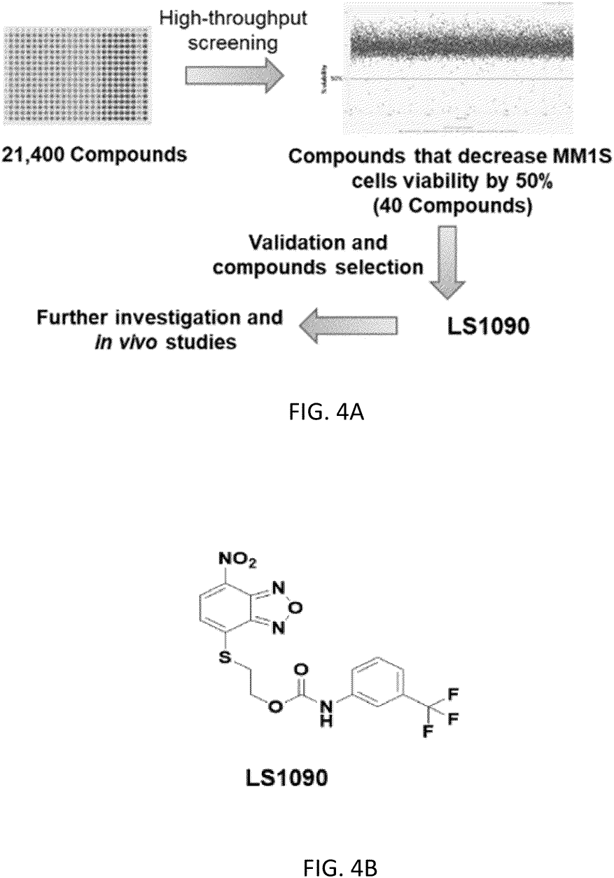

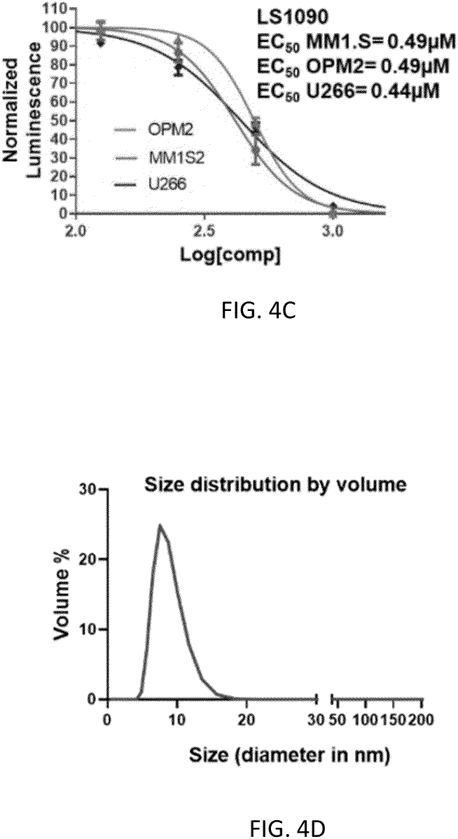

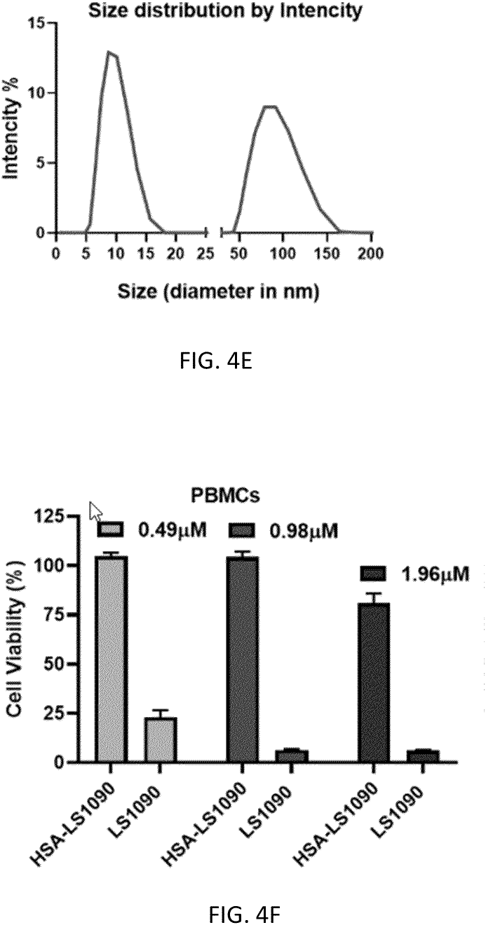

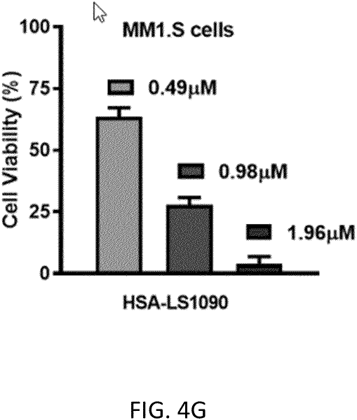

Unbiased cellular high throughput drug screening identifies LS1090 as a potential drug candidate for MM: A typical procedure to identify new drugs is to screen diverse libraries of compounds against a known target. In this study, an unbiased high-throughput cellular screening method and cell viability assay was utilized as the readout, where over 21,400 compounds from different small molecule libraries (Maybridge, NCI,NeXT Diversity 3500, and Diversity SAR 3500) were screened against a widely used human MM cell line, MM.1S (FIG. 4A). This assay identified LS1090 as a potential drug candidate (FIG. 4B). Unlike many other compounds in the libraries, LS1090 was effective in inhibiting the proliferation of many MM cell lines, with sub-micromolar concentrations at half-maximal response (EC₅₀) values for human MM.1S (0.49 µM), OPM2 (0.49 µM), and U266 (0.44 µM) cells (FIG. 4C). It was found that LS1090 also inhibited the growth of a mouse MM cell line, 5TGM1 (EC₅₀ = 0.32 µM). Encouraged by these data, the toxicity of LS1090 is selective for tumor cells was determined. Treatment of the nontumor PBMCs for 24 h with LS1090 exhibited significant cytotoxicity at the EC₅₀ and higher concentrations, possibly due to nonspecific internalization aided by the DMSO solution use to solubilize the compound.

Previous studies have shown that nanoparticles (NPs) can mitigate the toxic effects of potent drugs on healthy tissue and deliver a high payload of the compounds into tumors. In particular, the formulation of lipophilic drugs with HSA stabilized the compounds and enhanced their tumor retention, due in part to the rapid uptake of this protein by cancer cells. Consequently, LS1090 (2 mg) was mixed in an aqueous solution (10 mL) containing HSA (250 mg), unbound LS1090 was removed through size exclusion chromatography, and lyophilized the eluate to obtain HSA-LS1090 NPs. Over 99% of the total volume of the particles have an average hydrodynamic diameter of 8.41±2.01 nm NPs and polydispersity index (PDI) of 0.188 (FIG. 4D). Only about 0.1% of the total volume of the particles forms little aggregate of average diameter 75.63±21.68 nm that skewed the sigmoidal distribution yielding a z-average of 70.24 nm, visible in the intensity percentage distribution of the LDS measurements (FIG. 4E).

Next, if HSA-LS1090 NPs could exhibit selective cytotoxic effects on MM cells compared to PBMCs after 24 h incubation was investigated. The results showed that HSA-LS1090 did not alter the normal cell viability of PBMCs, exhibiting minimal effect at threefold the effective LS1090 EC₅₀ concentration (FIG. 4F). At 0.49 µM LS1090 concentration, HSA-LS1090 inhibited MM.1S growth by only about 20% and achieved the equivalent cytotoxicity of the free LS1090 about twice (0.98 µM) the EC₅₀ value. These differences could be attributed to a variety of factors, including the slow release of LS1090 from HSA NPs. This data suggest that the formulation of LS1090 in HSA is effective in inhibiting MM cells while sparing nontumor PBMCs, supporting the use of HSA-LS1090 for treating MM in tumor-bearing mice.

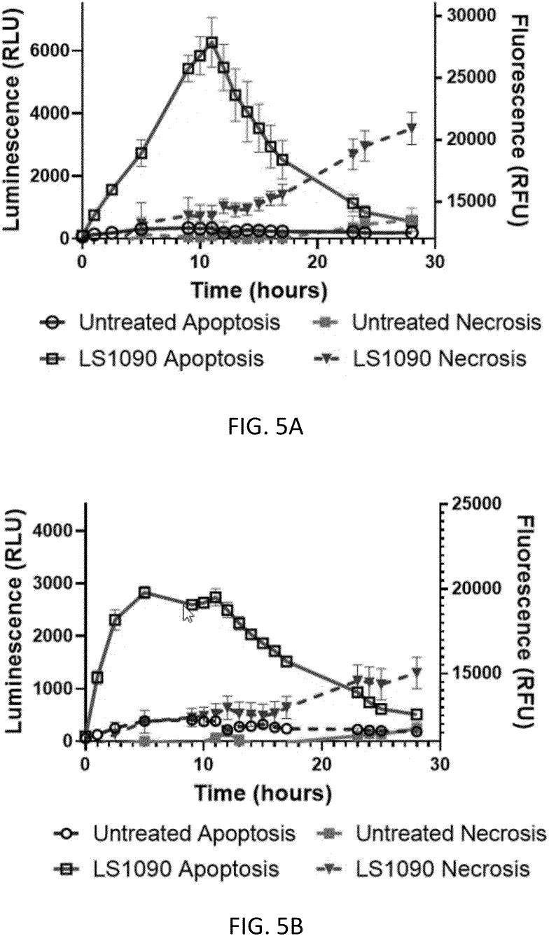

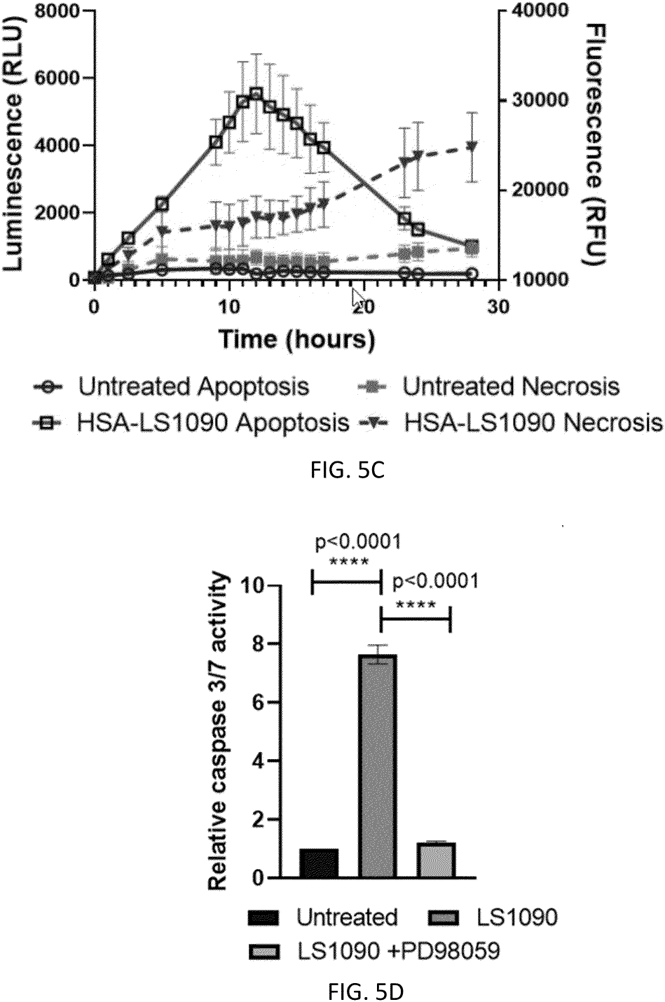

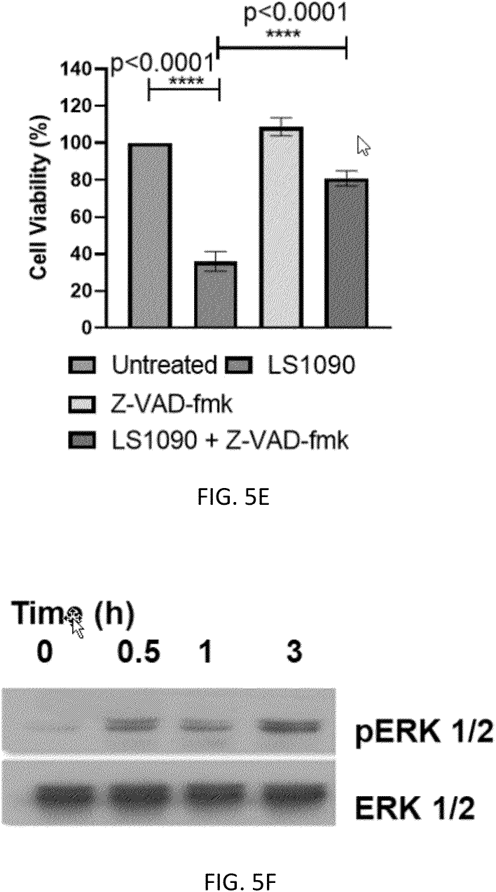

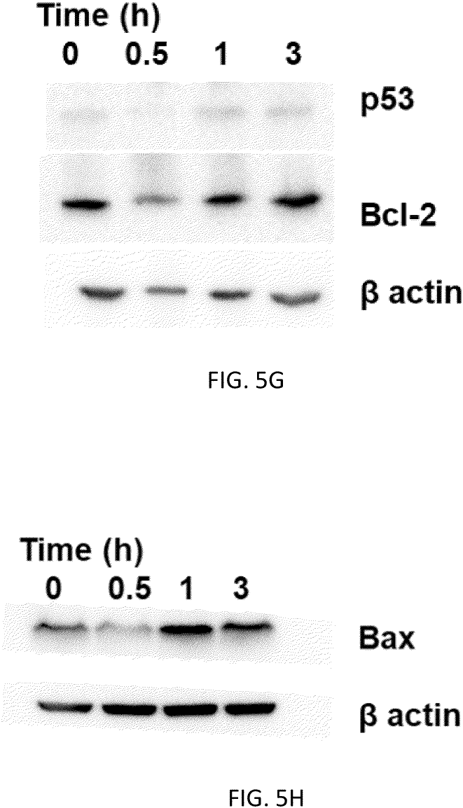

LS1090 activates ERK1/2 to induce apoptosis in MM cells via a p53-independent pathway: Chemotherapeutics exert their effects via multiple cell death pathways, including apoptosis and necrosis. To determine the dominant mechanism of LS1090-induced MM cell death, RealTime-Glo Annexin V Apoptosis and Necrosis Assay were used, which report both pathways simultaneously. The method reports apoptosis by detecting phosphatidylserine on the outer leaflet of cell membranes and necrosis by measuring plasma membrane integrity. Time-dependent measurements showed an early onset of apoptosis in MM cells treated with LS1090, peaking at about 10 h post-treatment before gradually decreasing toward baseline after 24 h (FIG. 5A). It was observed that necrosis lagged apoptosis, becoming the dominant cell death mechanism 24 h post-treatment. Relatively resistant another MM cell line U266 showed the same apoptosis-necrosis pattern when treated with LS1090 (FIG. 5B). Interestingly, the HSA-LS1090 exhibited a similar trend as the aqueous DMSO solution of the compound (FIG. 5C), indicating that the HSA formulation did not alter the mechanism of cell death. This delayed necrosis onset points to a cell death mechanism where pro-apoptosis induction transforms into secondary necrosis when phagocytes are not available to remove apoptotic cells. Given that apoptosis can be reversed, this study suggests that LS1090-induced apoptosis is likely irreversible.

ERK1/2 signaling plays an essential role in the cascade of apoptosis-mediated cell death. To interrogate its involvement, MM.1S cells were treated with LS1090 and Western blots used to determine ERK1/2 activation. Data analysis showed that LS1090 rapidly induced ERK1/2 phosphorylation in less than 1 h post-treatment, and the activated state was sustained over 3 h (FIG. 5F). Given that ERK1/2 signaling can utilize caspase-dependent or independent pathways to induce apoptosis, LS1090 mediated activation of caspase 3/7 in MM.1S cells was examined in the presence or absence of ERK1/2 activation inhibitor, PD98059. Whereas LS1090 triggered over seven-fold increase in caspase 3/7 activity after 4 h incubation, this process was inhibited in the presence of PD98059 (FIG. 5D). The consequential effect of caspase 3/7 activation on cell death was further validated by determining the viability of LS1090-treated MM1.S cells in the presence or absence of the caspase inhibitor, Z-VAD-fmk. This data showed that LS1090 eradicated 60% of the cells, but co-incubation with Z-VAD-fmk inhibited this event (p< 0.0001), confirming that LS1090 triggers caspase 3/7 mediated apoptosis (FIG. 5E).

ERK is known to exert its pro-apoptotic function by upregulating p53 and downregulating Bcl-2 expression. However, a similar association was not found in this study, evidenced by the lack of p53 enhancement in MM.1S cells after treatment with LS1090 (FIG. 5G). Similarly, LS1090 did not downregulate the anti-apoptotic protein, Bcl-2. In contrast, the expression of pro-apoptotic protein, Bax, increased within 1 h post-treatment (FIG. 5H). Collectively, these results demonstrate that LS1090 uses a p53-independent ERK1/2 signaling pathway via Bax axis to induce caspase 3/7 mediated apoptosis in MM.1S cells.

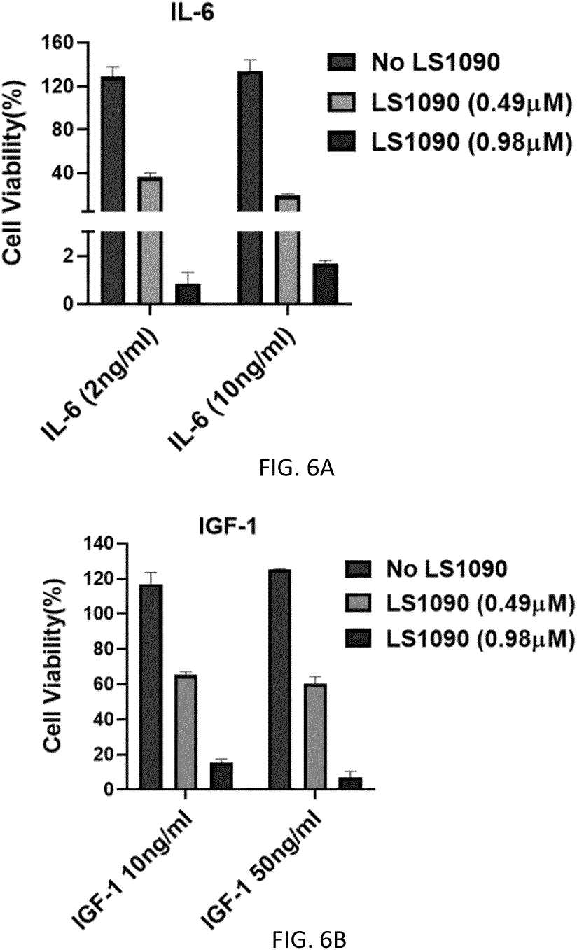

The induction of apoptosis in U266 MM cells, which express high levels of the anti-apoptotic proteins, suggests that LS1090 can have a pro-apoptotic effect on these chemoresistant cells. IL-6 and IGF-1 are major growth factors that protect MM cells from drug-induced apoptosis in vivo. Under conditions that simulated the expression of anti-apoptotic proteins, it was found that neither recombinant IL-6 nor IGF-1 blocked LS1090-triggered cytotoxicity in MM.1S cells (FIG. 6). This remarkable inhibitory effect indicates that LS1090 can overcome the protective effects of anti-apoptotic proteins in MM cells.

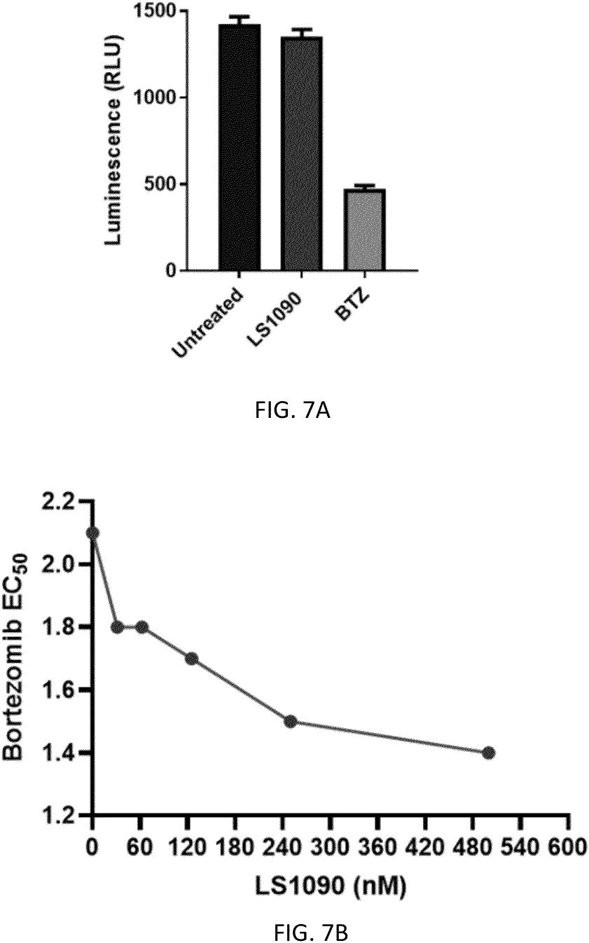

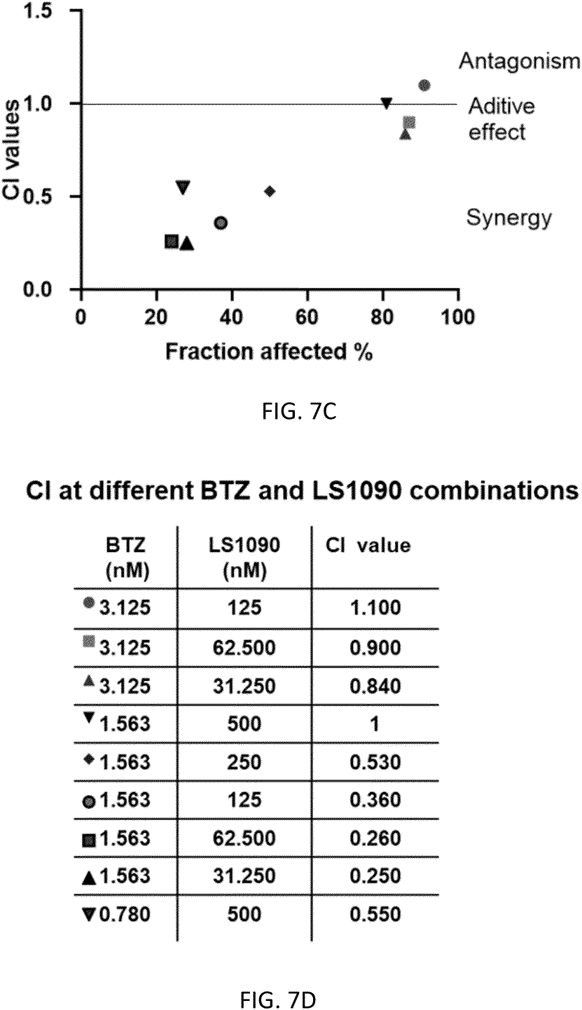

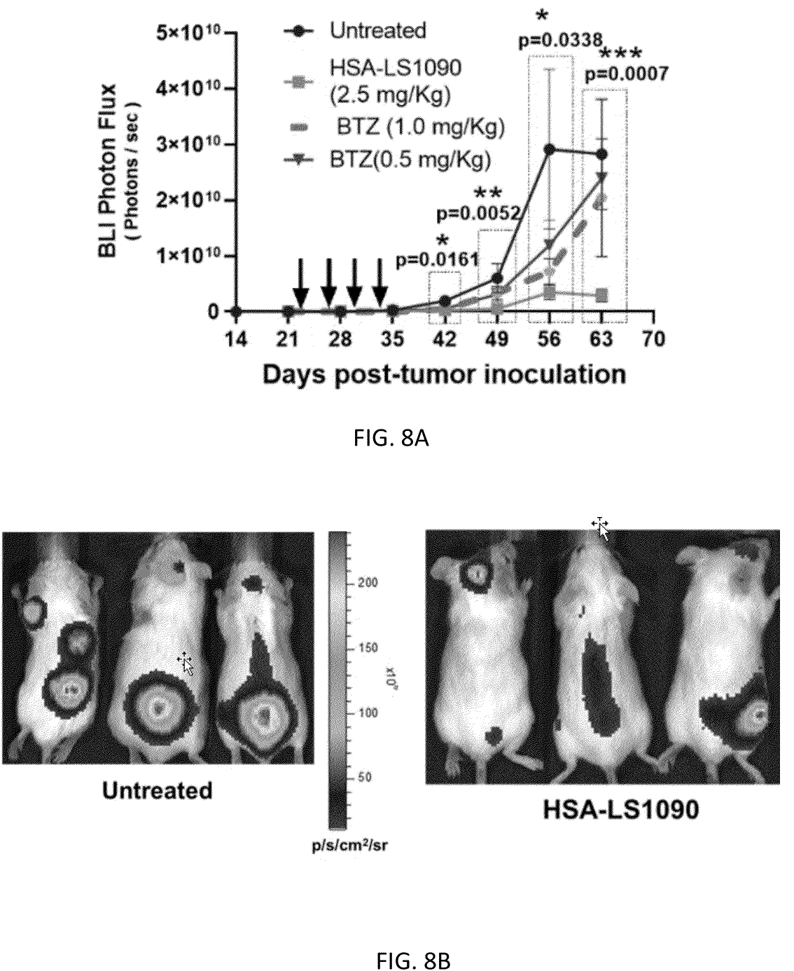

LS1090 synergistically enhances BTZ toxicity in vitro without inhibiting chymotrypsin-like activity: Proteasome inhibitors such as BTZ are widely used to treat MM, but the heterogeneous nature of this disease requires combination therapy to improve treatment response. Unfortunately, MM undergoes multiple cycles of relapses and remissions, with progressive resistance to standard therapies. As a result, new drugs that utilize complementary signaling pathways to kill MM cells would augment the response of refractory MM cells. BTZ inhibits chymotrypsin-like activity of proteasomes.A luminescence chymotrypsin-like assay confirmed that BTZ efficiently inhibits chymotrypsin-like activity in MM.1S cells at 0.1 µM, but LS1090 did not significantly perturb this enzyme activity after 4 h of incubation at ten-fold the concentration of BTZ (FIG. 7A).

Given that LS1090 and BTZ exert their cytotoxic effects by different pathways, it was postulated that their combination could enhance anti-MM activity. Firstly, it was found that increasing the concentration ratio of LS1090 to BTZ in MM.1S cells improved the EC₅₀ of BTZ from 2 nM to 1.4 nM (FIG. 7B). Although U266 cells are 10-fold less sensitive to BTZ than MM.1S cells due to the enhanced proteasome system activity, both MM cell lines exhibited similar EC₅₀ values after incubation with LS1090 (FIG. 4C). This result further supports that the LS1090 effect is less dependent on chymotrypsin-like activity. Secondly, different combinations of these drugs were tested and performed an isobologram analysis to determine how their interactions affect cell viability. It was demonstrated that the cytotoxic effects of BTZ and LS1090 in MM.1S cells are synergistic for the indicated concentrations at combination index (CI) of <1 (FIG. 7C and FIG. 7D), an outcome that could potentiate the therapeutic effects of BTZ in vivo.

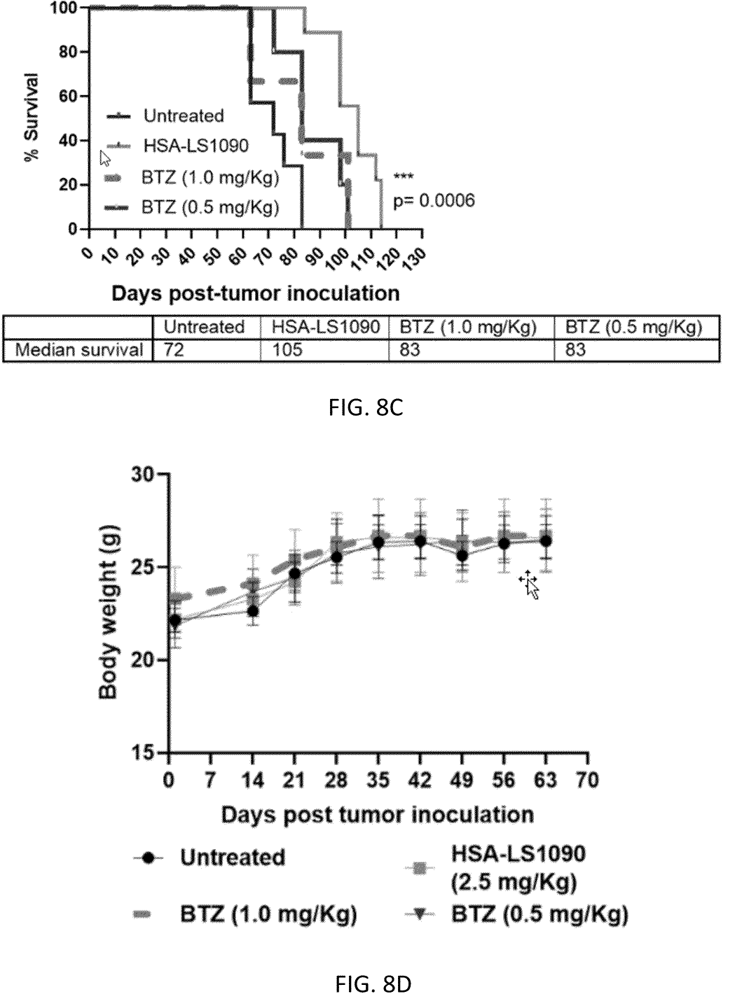

HSA-LS1090 inhibits MM proliferation in vivo and prolongs survival in murine model: Consistent with the pathophysiology of MM in patients, disseminated MM models of cancer are widely used to evaluate new drugs and treatment response.39 In this study, intravenous administration of MM1.S cells stably transfected with GFP and CBR Luciferase (MM1.S-GFP-Luc) in Fox Chase SCID beige mice developed a disseminated MM xenograft. After 15-21 days post-inoculation, bioluminescence imaging (BLI) revealed multiple sites of tumor proliferation in the mice. We initiated treatment with HSA-LS1090 (2.5 mg/kg effective dose of LS1090; n = 9 mice) when the whole body BLI photon flux reached a million photon counts per second, which was compared to the untreated group (n = 7 mice). Each treated mouse received two doses per week separated by 3 days over two weeks for a total of four dosages, and BLI was used to monitor tumor progression. The untreated group was injected with saline following the same treatment regimen of the HSA-LS1090. The endpoint for the study includes paralysis, loss of 20% of the original weight or sudden death due to the tumor burden before any other symptom appeared. A major challenge with using BLI as an endpoint in tumor progression study is the loss of signal linearity as more non-viable cell (necrotic) regions become dominant in the tumor mass, leading to therapy response-independent decrease in bioluminescence intensity. Given that over 40% of the untreated mouse group started exhibiting signal decrease by week 9 post-inoculation, we used this time point as an endpoint for the BLI monitoring of tumor progression. Our data show that HSA-LS1090 successfully inhibited tumor progression compared to the untreated control group by week 9 (FIG. 8A and FIG. 8B). The therapeutic effect of the HSA-LS1090 was visible from week 6 post-inoculation and significant statistical difference with the untreated group sustained until week 9. In addition to the loss in signal linearity, paralysis became a common occurrence in the untreated group after week 9. This demise was not observed in the treated cohort until about week 13. We found that HSA-LS1090 exerted a median survival benefit of 105 days compared to the 72 days for the untreated group (FIG. 8C).

Comparison of the therapeutic effects of HSA-LS1090 and BTZ in MM.1S-GFP-Luc tumor-bearing mice show that while the former achieved durable progression-free survival at 2.5 mg/kg, BTZ-treated mice at 0.5 mg/kg (n = 5 mice) or 1.0 mg/kg (n = 3 mice) experienced early remission and relapsed after 6-week post-inoculation when treatment was stopped (FIG. 8B). Attempts to increase BTZ dose to over 1 mg/Kg was unsuccessful due to the observed lethal effect on mice after the second dose. Collectively, our data demonstrate that HSA-LS1090 is a promising therapeutic compound and does not induce gross toxic effects relatively higher injected dose compared to BTZ, a standard of care drug for MM.

Conclusion: Prevention of frequent MM relapse remains the Holy Grail to improve treatment outcome and the quality of life. Toward this goal, we have identified a small drug candidate, LS1090, which inhibits MM proliferation by triggering caspase 3/7 apoptotic processes in MM cells. Not only did LS1090 exert its therapeutic effect in a p53-independent ERK1/2 signaling pathway via Bax axis, but the compound was also effective in the presence of MM growth factors (IL-6 and IGF-1), which are associated with chemoresistance and treatment failures.51 We further demonstrated that the combination of LS1090 with BTZ had a synergistic anti-MM activity in vitro, an outcome that could result from complementary chymotrypsin-like activity dependent and independent cell death pathways. Formulation of LS1090 in HSA produced well-defined NPs, which prevented toxicity to non-tumor PMBCs while efficiently inhibiting MM proliferation. In animal models of disseminated MM, HSA-LS1090 successfully inhibited tumor progression and extended survival by more than 30 days compared to the untreated controls. From mechanistic and safety to therapeutic efficacy considerations, we have uncovered a strategy to improve MM treatment and overcome therapy resistance by using LS1090 alone or in combination with other frontline chemotherapeutics.

### Materials and Methods

Cell culture and Reagents: Human MM cell lines MM1.S, U266, and OPM2 were cultured in RPMI-1640 medium supplemented with 10% fetal bovine serum, 100 units/mL penicillin, 100 µg/mL streptomycin at 37° C., 5% CO₂. PBMCs were purchased from Lonza Bioscience, USA and maintained overnight in RPMI-1640 medium supplemented with 10% fetal bovine serum, 100 units/mL penicillin, 100 µg/mL streptomycin at 37° C., 5% CO₂ before using for experiments. IL-6 and IGF-1 were purchased from R&D Systems. PD98059 was obtained from Cell Signaling Technology. Z-VAD-FMK was purchased from Santa Cruz. Bortezomib was obtained from Sigma. LS1090 [2-((7-nitrobenzo[c][1,2,5]oxadiazol-4-yl)thio)ethyl (3-(trifluoromethyl)phenyl)carbamate] was purchased from MolPort, Inc.M. Maybridge library was purchased from Thermo Fisher Scientific, NeXT Diversity 3500 and Diversity SAR 3500 sets were gifts from NCI.

High Throughput Screening: Screening was performed at the High Throughput Screening Center at Washington University using the following protocol. Pre-labeled Costar 384-well white clear bottom TC-treated plates were pre-spotted with 50 nL of 1 mM Maybridge, NeXT Diversity 3500 and Diversity SAR 3500 sets libraries compounds stock solutions (2 µM final concentration in PBS with 0.2% DMSO) and controls using the Hummingbird XL dispenser. PBS with 0.2% DMSO and 1 µM Bortezomib in PBS with 0.2% DMSO were used as negative and positive controls, respectively. MM1S cells at density of 5000 per well were pelted in 25 µL of media into compounds pre-spotted plates using a Multidrop384 and allowed to incubate for 30-45 min at room temperature prior to placing them in an incubator. Cells were incubated with compounds for 24 h at 37° C., 5% CO₂. After the incubation, the cell viability was assessed using the CellTiter-Glo assay (Promega). The assay was performed using the integrated and automated screening platform (Beckman Coulter) at the HTSC. SAMI EX software was used to design and execute the assay and enabled efficient and uniform assay execution across all the plates in a run. CellTiter Glo reagent (25 µL) equilibrated to room temperature was added using the Multidrop384 dispenser into cells that were pre-incubated at room temperature for 30 min. After incubating for 1 h at room temperature, the luminescence signal was read using the Envision plate reader (Perkin Elmer).

HSA-LS1090 synthesis and characterization: Sterile 25% HSA solution (1 mL) (GRIFOLS) was diluted with sterile water (9 mL) to obtain 2.5% HSA working solution (10 mL). 2.5 mg of LS1090 was dissolved in 25 µL DMSO and the DMSO solution was added to the HSA working solution under water bath sonication at 25° C. After the addition, the combined solution was mixed on IKA KS 130 basic plate shaker for 6 h at room temperature. The resulting mixture was purified by 5 K size-exclusion gravity column to eliminate any unbound LS1090 using microbiology grade pure water as eluent. The purified solution was lyophilized in Thermo Fischer SAVANT RVT5105lyophilizer to obtain the HSA-LS1090 NPs as dry powder. The lyophilized product was stored at 4° C. and reconstituted only prior to use in 0.9% saline. The concentration of LS1090 in the reconstituted solution was determined by UV analysis using the molar extinction coefficient of LS1090 (0.018 µM in ethanol, diluted 1000 times from DMSO stock solution). The concentration of the HSA in the reconstituted product was determined by Bio-Rad Quick Start Bradford Protein Assay kit. The hydrodynamic size and disparity of the particles in the solution were determined by dynamic light scattering (DLS) using Malvern Zetasizer nano Series.

Cell viability assay: Twenty thousand MM1.S, U266, OPM2, and PBMCs in 100 µL of growth media were added per well in a 96-well plate. The cells were incubated for 24 h with serial dilutions of LS1090 stock solution (10 mM in DMSO) or BTZ stock solution (1 mM in DMSO). Dilutions were prepared in growth media. Cells viability was assessed using the CellTiter-Glo luminescence-based assay (Promega). After adding 100 µL CellTiter-Glo reagent, relative luminescence units (RLU) were measured using the Synergy2 plate reader (BioTek) and expressed as a percentage of the untreated cell controls.

Apoptosis and caspase activation assays: Caspase3/7 activity was measured using Caspase-Glo3/7 Assay System (Promega). MM.1S cells were seeded in a white 96-well plate at a density of 20000 cells per well. Cells were pretreated with 20 µmol/L PD98059 followed by incubation with 1 µM LS1090 for 4 h at 37° C., 5% CO₂ before accessing caspase 3/7 activity. Caspase-Glo3/7 reagent was added directly to the cells and incubated for 30 min before recording luminescence using the Synergy2 plate reader (BioTek). Data were represented as a fold change from the untreated cells control. Kinetics of apoptosis and necrosis was monitored using RealTimeGlo Annexin V Apoptosis and Necrosis Assay (Promega). MM.1S cells were treated with 1 µM LS1090 and incubated in the presence of 100 µL the RealTimeGlo Annexin V Apoptosis and Necrosis reagent at 37° C., 5% CO₂. The luminescence and fluorescence signals were monitored for 24 h. Relative luminescence units (RLU) and relative fluorescence units (RFU) were measured at different time points using the Synergy2 plate reader (BioTek).

Proteasome activity assays: MM.1S cells were seeded in a white 96-well plate at a density of 20000 per well. Cells were treated with 1 µML LS1090 or 100 nM Bz for 4 h before accessing chymotrypsin-like activity using Proteasome-Glo™ Chymotrypsin-Like, (Promega). After adding the Proteasome-Glo™ reagent, luminescence was determined as relative light units (RLU) using the Synergy2 plate reader (BioTek).

Cell extract preparation and Western blotting: Protein extracts were prepared by washing cells with PBS (pH 7.4) and suspending in RIPA lysis buffer (20 mM Tris-HCI (pH 7.5), 150 mM NaCl, 1 mM Na₂EDTA, 1 mM EGTA, 1% NP-40) containing protease and phosphatase inhibitors (Sigma). Equal amounts of protein, as determined by the BCA protein assay (Thermo Fisher Scientific), were subjected to SDS-PAGE (12% gels; Bio-Rad), followed by transfer to nitrocellulose membrane. Membrane were incubated with 1:1000 anti-ERK1/2 (Thr202/Tyr204, anti-phosphoERK1/2 (Thr202/Tyr204), anti-p53, anti-Bcl2, anti-Bax (Cell Signaling Technology) and anti-βActin (Santa Cruz). Secondary antibodies consisted of horseradish peroxidase-conjugated goat anti-rabbit IgG or goat anti-mouse IgG (Sigma). Detection was done by the enhanced chemiluminescence method (Thermo Fisher Scientific).

Isobologram analysis: MM.1S cells were treated with 7 concentrations of LS1090 (1000 nM, 500 nM, 250 nM, 125 nM, 62 nM, 31.25 nM) each with combination with 7 concentrations of BZ (50 nM, 25 nM, 12.5 nM, 2.25 nM, 3.12 nM, 1.56 nM, 0.78 nM). Each drug was also used alone at these concentrations. Totally 49 different combinations representing 13 different ratios were tested. Cell viability was determined by CellTiter-Glo luminescence-based assay (Promega) as described above. Each data point was performed in triplicates. Isobologram analysis was performed using CompuSyn software (ComboSyn, Inc.) and combination index <1 indicates synergism.

In Vivo Study: All the studies were conducted in compliance with the Washington University Animal Welfare Committee’s requirements for the care and use of laboratory animals in research. Fox Chase SCID Beige mice (From in house colony) (8-10 weeks old) were implanted with MM1.S cells stably transfected with GFP and CBR Luciferase (MM1.S-GFP-Luc) (2 million cells in 100 µL DPBS per mouse) by tail vein injection. MM1.S-GFP-Luc cells were generously provided by Dr. Dipersio (Washington University School of Medicine). The progression of tumor was monitored by whole-body BLI once a week post tumor inoculation. Therapy was started after week 3 post inoculation when the whole body photon flux was 1x106 photons/sec. HSA-LS1090 was administered intravenously (IV) twice a week, 3 days apart, for two consecutive weeks. The Untreated groups received the vehicle saline IV following the same regimen. BTZ was administered by intraperitoneal (IP) injections following the same treatment regimen of HSA-LS1090.

### Example 4: Efficacy of Derivatives and on Various Cancer Cell Lines

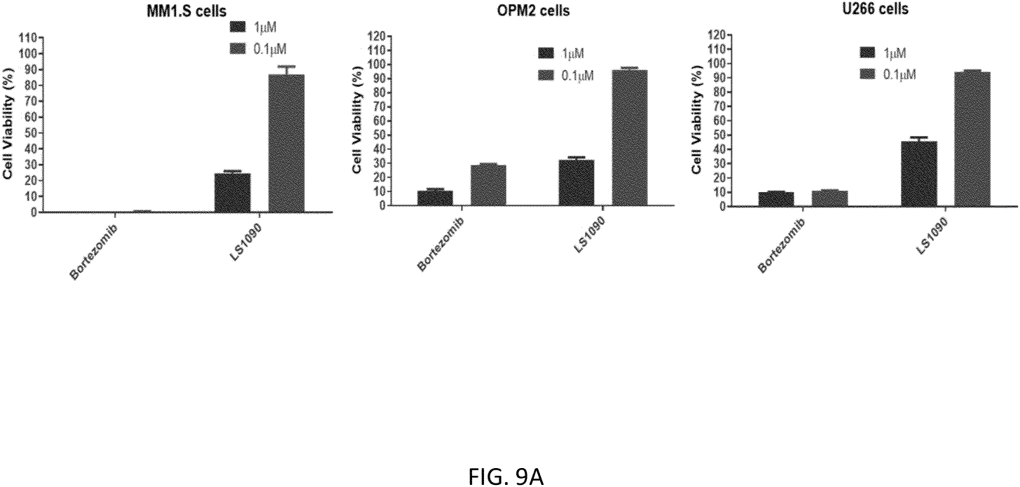

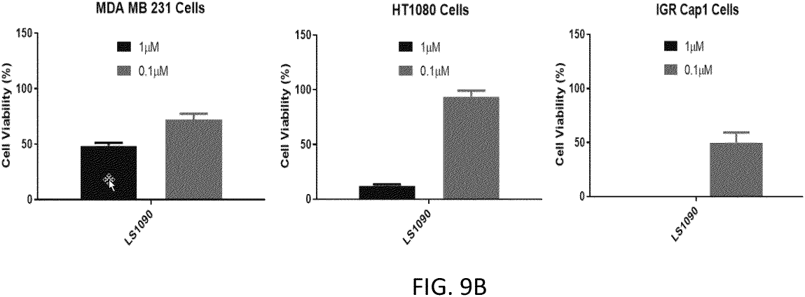

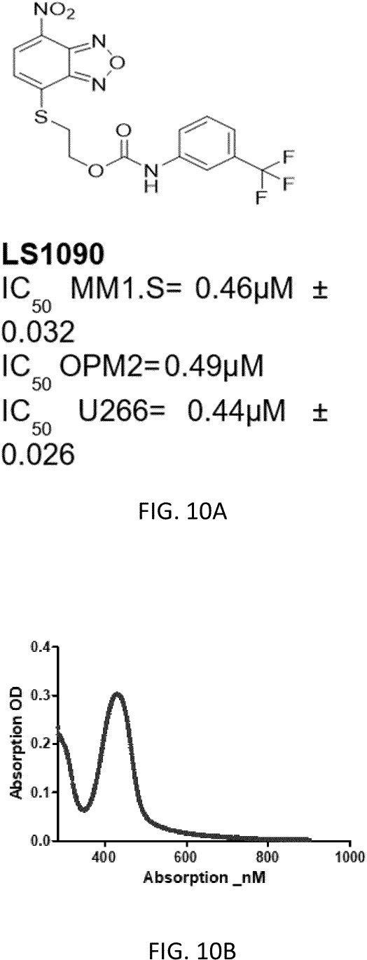

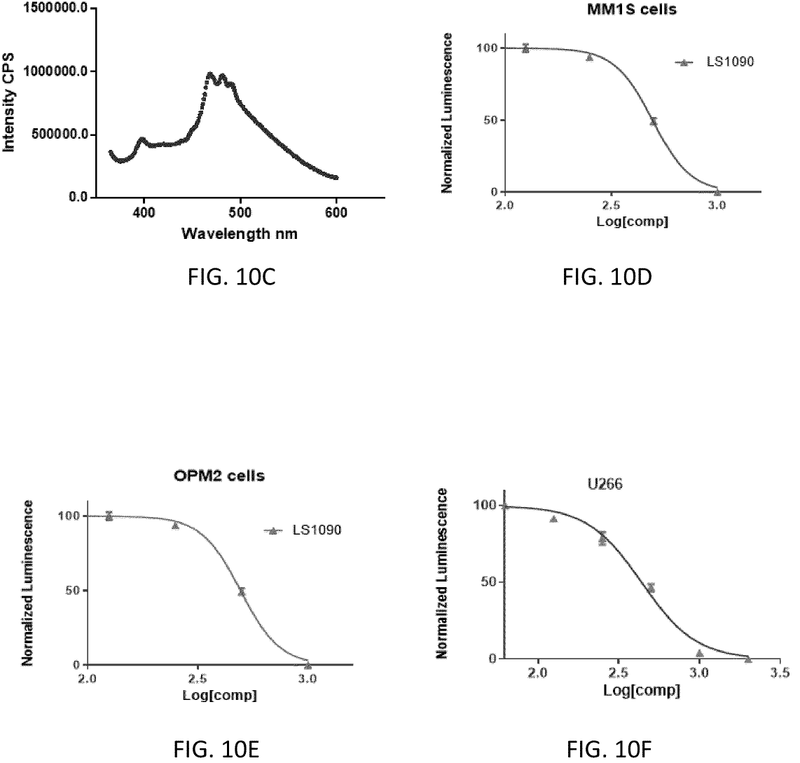

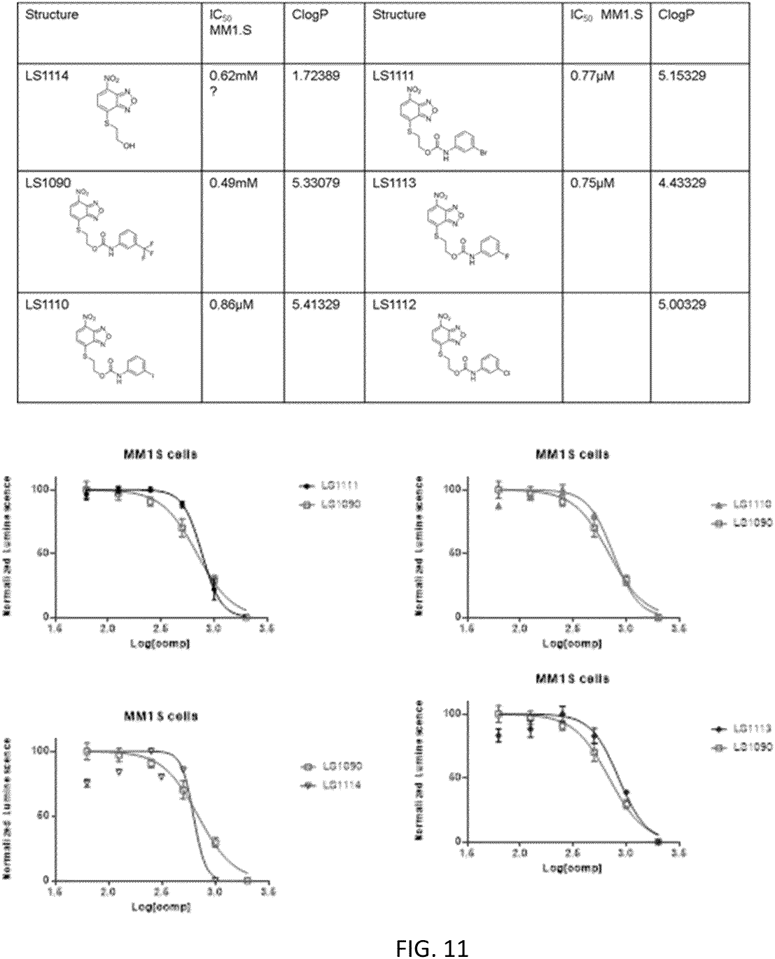

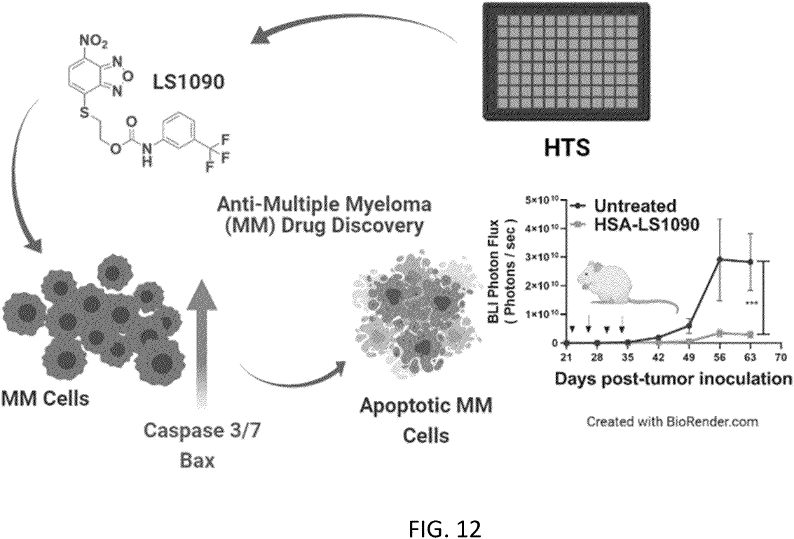

The IC50 values of were quantified for human MM1S,OPM2 and U266 cells, yielding a concentration of 0.49 µM for MM1.S and OPM2 and 0.44 µM for U266. Further, viability of human U266 cells was decreased 55% when incubated with 1 µM LS1090, further validating the sub-micromolar potency of LS1090 against multiple MM lines. IC₅₀ values were also quantified for the mouse MM cell line 5TGM, giving a value of 0.32 µM. Additionally, the viability of selected cancer cell lines were evaluated (IGR Cap1, HT1080, and MDA MB231) after 24 h incubation with LS1090 (see, e.g. FIGS. 9-11). LS1090 at 1 µM concentration had an effect on all tested cancer cell lines.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ± 20 %, preferably up to ± 10 %, more preferably up to ± 5 %, and more preferably still up to ± 1 % of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

It should also be understood that, unless clearly indicated to the contrary, in any methods claimed herein that include more than one step or act, the order of the steps or acts of the method is not necessarily limited to the order in which the steps or acts of the method are recited.

Terms used herein may be preceded and/or followed by a single dash, “—”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C1-C6alkoxycarbonyloxy and OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to CH₂—, CH₂CH₂—, CH₂CH₂CHC(CH₃)-, and CH₂CH(CH₂CH₃)CH₂—.

The term “alkylene” refers to a bivalent alkyl group. An “alkylene chain” is a polymethylene group, i.e., —(CH₂)n—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3 dihydrobenzofuran-4-yl, 2,3 dihydrobenzofuran-5-yl, 2,3 dihydrobenzofuran-6-yl, 2,3 dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The terms “cyano” and “nitrile” as used herein, mean a CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH2)w—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means Cl, Br, I or F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “oxo” as used herein means a =O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a =S group.

The term “unsaturated” as used herein means the referenced chemical structure contains at least one multiple carbon-carbon bond, but is not aromatic. For example, an unsaturated cycloalkyl group as defined herein includes cyclohexenyl, cyclopentenyl, cyclohexadienyl, and the like.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art. An effective amount is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder.

What is claimed is:

1. A compound of Formula (I) and salts thereof: 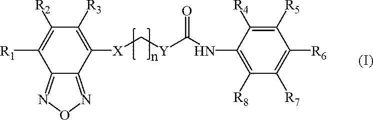
wherein X and Y are independently selected from O, S, NH Se, (OC₂H₄)₁₋₅;
wherein R¹, R², R³, R⁴, R⁵, R⁶, R⁷, and R⁸ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur; and
n is an integer from 1-5.

2. The compound of claim 1, wherein R₁ is NO_{2.}

3. The compound of claim 1 or claim 2, wherein R₂ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

4. The compound of any one of the preceding claims, wherein R₂ is hydrogen.

5. The compound of any one of the preceding claims, wherein R₃ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

6. The compound of any one of the preceding claims, wherein R₃ is hydrogen.

7. The compound of any one of the preceding claims, wherein R₄ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

8. The compound of any one of the preceding claims, wherein R₄ is hydrogen.

9. The compound of any one of the preceding claims, wherein R₅ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

10. The compound of any one of the preceding claims, wherein R₅ is hydrogen.

11. The compound of any one of the preceding claims, wherein R₆ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

12. The compound of any one of the preceding claims, wherein R₆ is hydrogen.

13. The compound of any one of the preceding claims, wherein R₇ is selected from hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

14. The compound of any one of the preceding claims, wherein R₇ is selected from a halogen, CH₃, CBr₃, and CCl₃.

15. The compound of any one of the preceding claims, wherein R₈ is selected from the group hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur.

16. The compound of any one of the preceding claims, wherein R₈ is hydrogen.

17. The compound of any one of the preceding claims, wherein X is selected from the group O, S, NH Se, (OC₂H₄)₁₋₅.

18. The compound of any one of the preceding claims, wherein Y is selected from the group O, S, NH Se, (OC₂H₄)₁₋₅.

19. The compound of claim 1, wherein the compound is selected from 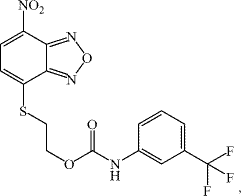 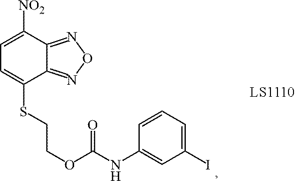 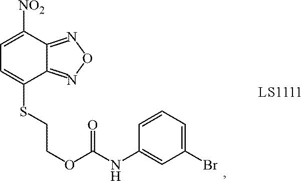 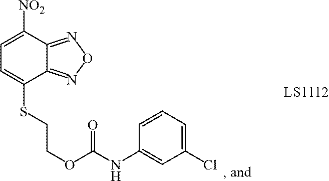 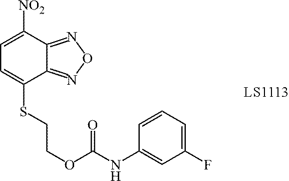 [SMILES: O=C(Nc1cccc(F)c1)OCCSc1ccc([N+](=O)[O-])c2nonc12]
.

20. A compound of Formula (II) and salts thereof: 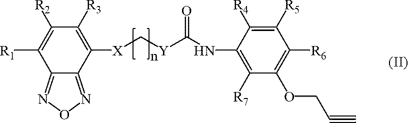
wherein X and Y are independently selected from O, S, NH Se, (OC₂H₄)_{1-5;}
wherein R₁, R₂, R₃, R₄, R₅, R₆, and R₇ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur; and
n is an integer from 1-5.

21. A compound of Formula (III) and salts thereof: 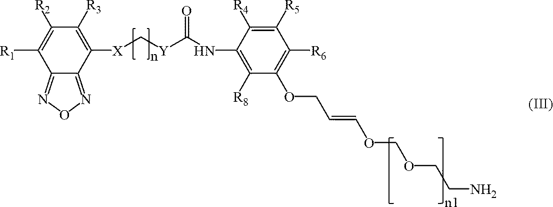
wherein X and Y are independently selected from O, S, NH Se, (OC₂H₄)_{1-5;}
wherein R₁, R₂, R₃, R₄, R₅, R₆, and R₇ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur;
n is an integer from 1-5; and
n1 is an integer from 1-5.

22. A compound of Formula (IV) and salts thereof: 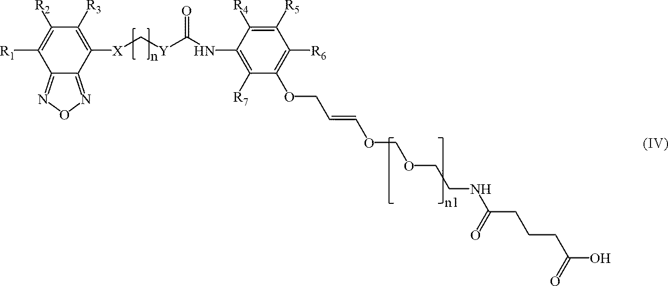
wherein X and Y are independently selected from O, S, NH Se, (OC₂H₄)_{1-5;}
wherein R₁, R₂, R₃, R₄, R₅, R₆, and R₇ are each independently selected from the group consisting of hydrogen, deuterium, halogen, CH₃, F, Cl, Br, OCH₃, OH, CN, NH₂, CH₂OH, CH₂NH₂, OCH₂CH₂N(CH₂H₅)₂, OCH₂CH₂N(CH₃)₂, CH₂OPO₃⁻², NHSO₂CH₃, CF₃, OCHF₂, —OCH₂CH₂NH₂, COOC₂H₅, COOH, COOCH₃, CF₃, CBr₃, CCl₃, NO₂, OEt, OtBu, OR′, SR′, NR′R′, NR′COR′, NR′CONR′R′, NR′CO₂R′, COR′, CO₂R′, NOR′, CONR′R′, OC(O)NR′R′, SO₂R′, SO₂NR′R′, NR′SO₂R′, NR′SO₂NR′R′, C(O)C(O)R′, C(O)CH₂C(O)R′, a substituted or unsubstituted C₁-C₆ alkyl, a substituted or unsubstituted C₁-C₆ alkenyl, a substituted or unsubstituted C₁-C₆ alkynyl, a substituted or unsubstituted aryl, and a substituted or unsubstituted heteroaryl; R′ is independently selected from the group consisting of hydrogen, substituted C₁-C₄ aliphatic moiety, aliphatic moiety containing nitrogen, oxygen, or sulfur, or alternately, two R′ moieties bound to the same nitrogen atom are optionally taken together with the nitrogen atom to form a 3-7 membered saturated or unsaturated ring having 1-2 additional heteroatoms independently selected from the group consisting of nitrogen, oxygen, or sulfur;
n is an integer from 1-5; and
n1 is an integer from 1-5.

23. A compound of Formula (II), (III), or (IV), wherein the compound is conjugated to a detectable label.

24. A pharmaceutically acceptable salt of a compound of any of the proceeding claims.

25. A pharmaceutical composition comprising a compound of any of the proceeding claims.

26. The pharmaceutical composition of claim 25, wherein the composition is formulated as a nanoparticle carrier for the compounds.

27. The nanoparticle composition of claim 26, wherein the nanoparticle is a liposome or protein nanoparticle.

28. A compound or composition of any of the preceding claims, wherein the compounds results in ERK1/2 activation to induce caspase 3/7-dependent apoptosis of a cancer or tumor.

29. A compound or composition of any of the preceding claims, wherein the compound results in increased expression of pro-apoptotic protein Bax in a cancer or tumor cell.

30. A method of a treating tumor or cancer in a subject in need thereof, the method comprising administering a therapeutically effective amount of a pharmaceutical composition comprising one or more compounds of claims 1-30 to the subject.

31. A method of killing a tumor or cancer cell in need thereof, the method comprising administering a therapeutically effective amount of a pharmaceutical composition comprising one or more compounds of claims 1-30 to the subject.

32. The method of claim 30 or claim 31, further comprising administering an additional cancer therapy.

33. The method of claim 32, wherein the additional therapeutic agent is bortezomib.

34. A method of treating a tumor or cancer in subject in need thereof, wherein the tumor or cancer is resistant to apoptosis, the method comprising the method comprising administering a therapeutically effective amount of a pharmaceutical composition comprising one or more compounds of claims 1-22.

35. A method of treating cancer in a subject in need thereof, the method comprising administering a therapeutically effective amount of a pharmaceutical composition comprising one or more compounds of claims 1-22 to the subject, wherein the cancer is selected from hematological tumors such as leukemia, lymphoma and multiple myeloma, and solid tumors such as lung cancer, liver cancer, pancreatic cancer, CNS cancers, breast cancer, ovarian cancer, colon cancer, renal cancer, melanoma, prostate cancer and head and neck cancers.

36. A method of inhibiting cancer stem cell growth and/or differentiation, the method comprising contacting the stem cell with a composition comprising one or more compounds of claims 1-22.

37. A method of treating a cancer or tumor comprising, administering a therapeutically effective amount of a pharmaceutical composition comprising one or more compounds of claims 1-22 and an excitation source such that a singlet oxygen is generated in the cancer or tumor cell.

38. A method of detecting or imaging a tumor or a cancer in a subject in need thereof, the method comprising administering a composition comprising a compound of claim 23, and imaging the detectable label thereby detecting or imaging the cancer or tumor in a subject.
